(19) United States
(12) Patent Application Publication
(10) Pub. No.: US 2010/0190739 A1
(43) Pub. Date: Jul. 29, 2010

(54) Rapidly Dissolving Vitamin Formulation and Methods of Using the Same

(75) Inventors: Angela Sutterer, Ballwin, MO (US); Suzanne M. Yachechak, Hackensack, NJ (US)

Correspondence Address: SENNIGER POWERS LLP, 100 NORTH BROADWAY, 17TH FLOOR, ST LOUIS, MO, 63102, US

(73) Assignee: FLEMING AND COMPANY, PHARMACEUTICALS, Fenton, MO (US)

(21) Appl. No.: 12/638,629

(22) Filed: Dec. 15, 2009

Related U.S. Application Data
(60) Provisional application No. 61/122,615, filed on Dec. 15, 2008.

(51) Int. Cl. A61K 31/714; A61P 3/02

(52) U.S. Cl. 514/52

(57) ABSTRACT

Rapidly dissolvable nutritional compositions and methods of providing nutritional supplementation to candidates for, patients undergoing, or patients having undergone bariatric surgery, to patients suffering renal disease, and to candidates for or patients undergoing dialysis. The compositions generally comprise one or more vitamins and optionally minerals and dissolve in less than about 90 seconds upon oral administration.

### CROSS-REFERENCE TO RELATED APPLICATIONS

This application claims the benefit of U.S. Provisional Application Ser. No. 61/122,615 filed Dec. 15, 2008, the entire content of which is hereby incorporated herein by reference.

### FIELD OF THE INVENTION

The present invention generally relates to rapidly dissolvable nutritional compositions and methods of providing nutritional supplementation to candidates for, patients undergoing, or patients having undergone bariatric surgery, to patients suffering renal disease, and to candidates for or patients undergoing dialysis.

### BACKGROUND

The post-surgical dietary restrictions and malabsorption issues related to the variety of bariatric weight loss surgery procedures available can lead to significant nutrient deficiency. In particular, patients having undergone bariatric surgery have very restricted dietary intake for approximately six months following surgery. Thus, regardless of the type of surgery (e.g., gastric banding or gastric bypass surgery), patients are at an increased risk of nutritional deficiencies due to the changes in food and liquid intake, digestive anatomy (i.e., changes to the size of or constrictions in the stomach (e.g., a reduction of the “usable” stomach volume to about 5-10% of the original stomach volume) and intestines), and the increased occurrence of persistent patient vomiting. As a result, bariatric patients are commonly deficient in the water soluble vitamins, and in particular the B vitamins (such as B₁, B₂, B₃, B₅, B₆, B₇, B₁₂, and folic acid) and vitamin C, fat soluble vitamins (such as vitamins A, D, and E), and essential minerals (such as iron, selenium, copper, manganese, molybdenum, calcium, magnesium, and zinc).

Because of these post-operative issues, bariatric patients are often unable to meet necessary nutritional requirements through simple food consumption alone. Instead, vitamin and mineral supplementation is required. However, swallowing supplement pills or tablets is not necessarily feasible either, as the supplement occupies a significant portion of the size-limited stomach, thereby further constricting food and liquid intake at a time when the ability to intake even small amounts of food and water is already substantially restricted due to the size reduction of the digestive anatomy. Because of the size of the typical supplement tablet relative to the size-limited digestive anatomy, typical nutritional supplementation may also cause nausea or vomiting. In order to more easily swallow nutritional supplements, some amount of liquid is typically required. This liquid also occupies additional volume in the patient's size-limited stomach. Moreover, a patient's perception of flavors may also be altered from their pre-surgical state, thereby making typical liquids used to swallow pills, such as juices and sodas, taste unpleasant. Thus, patient compliance with a necessary nutritional supplement regimen is challenging.

Accordingly, a need persists for a nutritional supplement method and composition for effective treatment of bariatric patients to satisfy the nutritional, physical, and emotional needs of the patients.

Patients suffering from renal disease, and in particular those undergoing dialysis, are also at an increased risk of nutritional deficiencies. In particular, individuals with renal disease often lack essential B-vitamins and vitamin C. This may occur, for example, as a result of a loss of appetite, required dietary restrictions, medications that interfere with vitamin absorption, and removal of these particular water soluble vitamins from the blood by required dialysis treatments.

Most commonly, the loss of these essential vitamins results from the required dietary restrictions. In particular due to the patients' diminished kidney function, they are forced to modify their diets to greatly reduce their intake of potassium, phosphorus, sodium, and protein. This modification is required, as consumption of potassium in the typical daily dietary amount could cause heart damage and arrest, consumption of phosphorus in the typical daily dietary amount could cause an unhealthy reduction in calcium in the blood, and consumption of sodium in the typical daily dietary amount could increase water retention and cause patient thirst, thereby jeopardizing patient compliance with the required fluid restrictions.

The required dialysis treatments also deplete the patient of essential B-vitamins and vitamin C, as these vitamins may be removed from the blood during dialysis. Thus, even if patients are capable of meeting their vitamin requirements through the consumption of food (in spite of the dietary restrictions), the dialysis treatment will often deplete the patient of vitamins B and C such that supplementation of these vitamins is necessary.

Because of these issues, renal and dialysis patients are often unable to meet the necessary nutritional requirements through simple food consumption alone. Instead, vitamin, and optionally vitamin and mineral, supplementation is often required. However, swallowing the typical supplement pills or tablets is not necessarily feasible either, as administration of such pills or tablets requires some amount of liquid. As elimination of water from the body is a function performed almost exclusively by the kidneys, fluid restrictions are also a requirement for renal and dialysis patients in order to avoid placing a significant strain on patients' already poorly or non-functioning kidneys. Thus, patient compliance with the typical necessary nutritional supplement regimen, which would generally require taking supplement pills with some volume of fluid, presents a challenge.

Accordingly, a need also persists for a nutritional supplement method and composition for effective treatment of renal and dialysis patients to satisfy the nutritional, physical, and emotional needs of these patients.

### SUMMARY OF THE INVENTION

In accordance with the present invention, a rapidly dissolvable nutritional supplement composition has been developed as a quick dissolve tablet with a smooth mouth feel that disintegrates in less than about 90 seconds without the need for contemporaneous administration of exogenous liquids. Moreover, the vitamin taste, as well as the mineral taste if minerals are present, is masked with flavor and sweetener. The nutritional composition of the present invention avoids or alleviates problems associated with conventional nutritional supplement pills or tablets and, therefore, results in improved patient compliance with and effectiveness of a post-operative nutritional supplement regimen or a renal or dialysis regimen.

Among the various aspects of the present invention are a rapidly dissolvable tablet for oral administration to a patient having undergone bariatric surgery and a method for providing nutritional supplementation to a bariatric patient utilizing such a tablet.

Briefly therefore, one aspect of the present invention is directed to a method for providing nutritional supplementation to a patient having undergone bariatric surgery. The method comprises orally administering to the patient a rapidly dissolving tablet, the tablet comprising; an active ingredient component comprising one or more vitamins or minerals; a disintegrant; and a directly-compressible, water-soluble carbohydrate; wherein the tablet dissolves in the mouth of the patient within about 90 seconds after administration of the tablet. In a particular embodiment, the active ingredient component comprises a mixture of two or more B vitamins selected from the group consisting of B₁, B₂, B₃, B₅, B₆, B₇, B₉, and B₁₂; one or more non-B vitamins; and one or more minerals.

Another aspect of the present invention is a method for providing nutritional supplementation to a patient having undergone bariatric surgery. The method comprises orally administering to the patient a rapidly dissolving tablet, the tablet comprising an active ingredient component comprising a mixture of two or more B vitamins selected from the group consisting of B₁, B₂, B₃, B₅, B₆, B₇, B₉, and B₁₂; one or more non-B vitamins; and one or more minerals, the active ingredient component being substantially free of therapeutic quantities of calcium and/or magnesium; a disintegrant; and a directly-compressible, water-soluble carbohydrate; wherein the tablet dissolves in the mouth of the patient within about 90 seconds after administration of the tablet; orally administering to the patient a second tablet, the tablet comprising therapeutic quantities of calcium and/or magnesium.

Another aspect of the present invention is a method for providing nutritional supplementation to a candidate for bariatric surgery. The method comprises orally administering to the candidate a rapidly dissolving tablet, the tablet comprising an active ingredient component comprising one or more vitamins or minerals; a disintegrant; and a directly-compressible, water-soluble carbohydrate; wherein the tablet dissolves in the mouth of the patient within about 90 seconds after administration of the tablet. In a particular embodiment, the active ingredient component comprises a mixture of two or more B vitamins selected from the group consisting of B₁, B₂, B₃, B₅, B₆, B₇, B₉, and B₁₂; one or more non-B vitamins; and one or more minerals.

Another aspect of the present invention is a rapidly dissolving tablet for oral administration for use in providing nutritional supplementation to a patient having undergone bariatric surgery. The tablet comprises an active ingredient component comprising a mixture of two or more B vitamins selected from the group consisting of B₁, B₂, B₃, B₅, B₆, B₇, B₉, and B₁₂; one or more non-B vitamins; and one or more minerals; a disintegrant; and a directly-compressible, water-soluble carbohydrate; wherein the tablet dissolves in the mouth of the patient within about 90 seconds after administration of the tablet.

Another aspect of the present invention is a rapidly dissolving tablet for oral administration for use in providing nutritional supplementation to a patient having undergone bariatric surgery. The tablet comprises an active ingredient component comprising a mixture of two or more B vitamins selected from the group consisting of B₁, B₂, B₃, B₅, B₆, B₇, B₉, and B₁₂; one or more non-B vitamins; and one or more minerals, the active ingredient component being substantially free of therapeutic quantities of calcium and/or magnesium; a super disintegrant; and a directly-compressible, water-soluble mannitol; wherein the tablet is sugar free.

Also among the various aspects of the present invention is a rapidly dissolvable tablet for oral administration to a renal patient or a patient undergoing dialysis and a method for providing nutritional supplementation to a renal or dialysis patient utilizing such a tablet.

Briefly, therefore, one aspect of the present invention is directed to a method for providing nutritional supplementation to a renal or dialysis patient. The method comprises orally administering to the patient a rapidly dissolving tablet, the tablet comprising an active ingredient component comprising one or more vitamins; a disintegrant; and a directly-compressible, water-soluble carbohydrate; wherein the tablet dissolves in the mouth of the patient within about 90 seconds after administration of the tablet. In a particular embodiment, the active ingredient component comprises a mixture of two or more B vitamins selected from the group consisting of B₁, B₂, B₃, B₅, B₆, B₇, B₉, and B₁₂; and one or more non-B vitamins. In a particular embodiment, the one or more non-B vitamins is vitamin C.

Another aspect of the present invention is a method for providing nutritional supplementation to a renal or dialysis patient. The method comprises orally administering to the patient a rapidly dissolving tablet, the tablet comprising an active ingredient component comprising a mixture of two or more B vitamins selected from the group consisting of B₁, B₂, B₃, B₅, B₆, B₇, B₉, and B₁₂, and one or more non-B vitamins, the active ingredient component being substantially free of phosphorous, potassium, and/or sodium; a disintegrant; and a directly-compressible, water-soluble carbohydrate; wherein the tablet dissolves in the mouth of the patient within about 90 seconds after administration of the tablet. In a particular embodiment, the one or more non-B vitamins is vitamin C.

Another aspect of the present invention is a method for providing nutritional supplementation to a candidate for dialysis. The method comprises orally administering to the candidate a rapidly dissolving tablet, the tablet comprising an active ingredient component comprising one or more vitamins; a disintegrant; and a directly-compressible, water-soluble carbohydrate; wherein the tablet dissolves in the mouth of the patient within about 90 seconds after administration of the tablet. In a particular embodiment, the active ingredient component comprises a mixture of two or more B vitamins selected from the group consisting of B₁, B₂, B₃, B₅, B₆, B₇, B₉, and B₁₂; and one or more non-B vitamins. In a particular embodiment, the one or more non-B vitamins is vitamin C.

Another aspect of the present invention is a rapidly dissolving tablet for oral administration for use in providing nutritional supplementation to a renal or dialysis patient. The tablet comprises an active ingredient component comprising a mixture of two or more B vitamins selected from the group consisting of B₁, B₂, B₃, B₅, B₆, B₇, B₉, and B₁₂, and one or more non-B vitamins; a disintegrant; and a directly-compressible, water-soluble carbohydrate; wherein the tablet dissolves in the mouth of the patient within about 90 seconds after administration of the tablet. In a particular embodiment, the one or more non-B vitamins is vitamin C.

Another aspect of the present invention is a rapidly dissolving tablet for oral administration for use in providing nutritional supplementation to a renal or dialysis patient. The tablet comprises an active ingredient component comprising a mixture of two or more B vitamins selected from the group consisting of B₁, B₂, B₃, B₅, B₆, B₇, B₉, and B₁₂, and one or more non-B vitamins, the active ingredient component being substantially free of phosphorous, potassium, and/or sodium; a super disintegrant; and a directly-compressible, water-soluble carbohydrate; wherein the tablet is sugar free. In a particular embodiment, the one or more non-B vitamins is vitamin C.

### BRIEF DESCRIPTION OF THE DRAWINGS

FIG. 1 is a schematic flow diagram illustrating a process for the preparation of the rapidly dissolving nutritional supplement tablet described herein.

FIG. 2 graphically depicts the results of the tablet hardness test in Examples 1 and 2.

FIG. 3 graphically depicts the results of the oral disintegration test in Examples 1 and 2.

FIG. 4 graphically depicts the results of the friability test in Examples 1 and 2.

FIG. 5 graphically depicts the results of the shipping test in Examples 1 and 2.

FIG. 6 graphically depicts the results of the tablet hardness over time test in Example 3.

FIG. 7 graphically depicts the results of the simulated patient test in Example 3.

FIG. 8 graphically depicts the hardness of tablets produced during a production run according to Example 3.

FIG. 9 graphically depicts the weight of tablets produced during a production run according to Example 3.

FIG. 10 graphically depicts the thickness of tablets produced during a production run according to Example 3.

### DETAILED DESCRIPTION OF THE INVENTION

The present invention is based in part on the observation that administration of nutritional supplements to bariatric patients (sometimes referred to herein as “patients having undergone bariatric surgery” or “bariatric surgery patients”) poses unique problems in the first several months following bariatric surgery. In particular, dietary restrictions and malabsorption issues, caused in large part by the substantial decrease in the size of the digestive anatomy, create unique issues related to nutritional supplementation for bariatric surgery patients. In addition to, and in part as a result of, these complications related to nutritional supplementation and administration, patient compliance with prior known nutritional supplementation regimens is also undermined, resulting in the potential for inconsistent and ineffective nutritional supplementation.

The present invention is also based in part on the observation that administration of nutritional supplements to renal patients and dialysis patients (sometimes referred to herein as “patients undergoing dialysis” or “hemodialysis patients”) pose similar problems. In particular, dietary modifications or restrictions, vitamin malabsorption issues due to administration of other medications, and loss of vitamins to dialysis treatments create unique issues related to nutritional supplementation for renal and dialysis patients. In addition to, and in part as a result of, these complications related to nutritional supplementation and administration, patient compliance with prior known nutritional supplementation regimens is also undermined, resulting in the potential for inconsistent and ineffective nutritional supplementation.

In order to provide bariatric patients with a more effective nutritional supplementation post-surgery, and to provide renal and dialysis patients with more effective nutritional supplementation, particularly during dialysis, the present invention is directed to methods for providing nutritional supplementation and maintaining necessary vitamin serum levels in these patients by orally administering to the patient a rapidly dissolvable tablet, preferably in the absence of concurrent oral administration of a liquid to the patient. The nutritional supplement tablet comprises an active ingredient component comprising, in one embodiment, a mixture of two or more B vitamins, and one or more non-B vitamins. In another embodiment, particularly suitable for bariatric patients, the active ingredient component also comprises one or more minerals. The tablet also comprises a disintegrant or a super disintegrant, and a directly compressible, water-soluble carbohydrate. Such directly compressible carbohydrates are well known in the art, and have flow and compression characteristics that make them compatible for use in high speed tableting processes without further augmentation or adjustments to the carbohydrates or the tableting processes (e.g., without the addition of a glidant, such as, for example, silicon dioxide) to increase flowability of the carbohydrate. Thus, generally such carbohydrates will be free flowing, allowing for rapid and homogenous flow of the carbohydrate during die filling and preventing common problems attributed to improper powder flow, such as non-uniformity in blending, under/over dosage of active ingredients, and inaccurate (e.g., incomplete or over) die filling. Typically, such a directly compressible carbohydrate will also have excellent compressibility with minimal to nonexistent elastic recovery, causing the tableted mass to remain in its compressed form once pressure is removed. Typically, the tablet will also contain a flavor component and a sweetener component in order to mask any unpleasant flavors associated with the active ingredient component, as well as other excipients used to provide the tablet with acceptable organoleptic properties (e.g., a pleasant mouth feel, tablet texture, tablet taste, etc.). Typically, oral administration of the tablet will result in the tablet dissolving upon contacting saliva in the mouth of the patient within about 90 seconds or less, thereby making the active ingredients in the tablet available for absorption in the digestive system of the patient almost immediately. As a result, administration of vitamins and minerals to the bariatric patient is achieved without occupying a substantial portion of the patient's reduced stomach volume or any portion of the patient's reduced digestive anatomy. Similarly, administration of vitamins and minerals to the renal or dialysis patient is achieved without substantially affecting the fluid restriction requirements of the patient. Again, these benefits are advantageously achieved, whether with respect to the bariatric patient or the renal or dialysis patient, without the typically requisite administration of a liquid in conjunction with the administration of the tablet.

Patients

The present nutritional composition and methods of nutritional supplementation were developed and are particularly suitable for patients having a limited digestive anatomy, such as for example those having recently undergone bariatric surgery (e.g., gastric banding or gastric bypass surgery), as well as for those on restrictive diet or fluid regimens, such as, for example, renal or dialysis patients.

Typically, bariatric patients are at an increased risk of nutritional deficiencies due to the changes in digestive anatomy (i.e., changes to the size of or constrictions in the stomach and intestines) and the increased occurrence of persistent patient vomiting. Not only do these factors cause an increase in risk of nutritional deficiencies, but they also make conventional forms of nutritional supplementation inefficient and sometimes simply physically or feasibly impossible to utilize. As a result, bariatric patients are often deficient in the water soluble vitamins, and in particular the B vitamins (such as B₁, B₂, B₃, B₅, B₆, B₇, B₁₂, and folic acid) and vitamin C, fat soluble vitamins (such as vitamins A, D, and E), and essential minerals (such as iron, selenium, copper, calcium, magnesium, manganese, molybdenum, and zinc).

Renal patients, and in particular those undergoing dialysis, are at an increased risk of nutritional deficiencies due to the substantial dietary restrictions required as a result of their compromised or nonexistent kidney function. In particular, because of the limits on foods that can be eaten (i.e., minimizing the consumption of foods having high concentrations of potassium, phosphorous, sodium, and/or protein), renal and dialysis patients often encounter vitamin, and in particular vitamin B and vitamin C, deficiencies. Moreover, dialysis often removes water soluble vitamins, and in particular the B vitamins and vitamin C, from the blood of the dialysis patients, thereby requiring constant vitamin replenishment or supplementation. This supplementation is further complicated by the fact that many renal and all and dialysis patients are also greatly limited in the amount of fluids they can consume on a daily basis.

Generally, therefore, the present methods and nutritional supplement compositions are particularly suitable for bariatric, renal, and dialysis patients as an alternative to nutritional supplementation in which a conventional nutritional supplement composition is administered in the form of a hard pill or tablet not readily dissolvable in the mouth of a patient, and in particular, such a conventional pill or tablet that cannot be easily swallowed without the concurrent administration of or in conjunction with a liquid. An example of such a patient is a person that has recently undergone some form of bariatric surgery, such as, for example, bypass surgery, such as gastric bypass surgery, wherein the stomach is reduced in size and food from the minimized stomach is forced to bypass part of the small intestine (an example of which is Roux-en-Y gastric bypass), gastric banding surgery, such as laparoscopic adjustable gastric banding or vertical banded gastroplasty, wherein an adjustable band is place around a portion of the stomach to restrict the efflux, and therefore, limit the influx, of food, or some combination of the two. Another example of such a patient is a renal patient having compromised kidney function or complete kidney failure, and in particular, such a patient undergoing kidney dialysis. Because of the limit on absorption, intake, or both, the vitamin compositions and methods of administration utilizing the same are of benefit to such patients.

Accordingly, the treatment methods disclosed herein are applicable for nutritional supplementation and maintenance of vitamin blood serum levels in a patient following bariatric surgery, including but not limited to gastric bypass or gastric banding, to renal patients, and to patients undergoing dialysis, and in particular such patients in need of vitamin B supplementation and/or maintenance, and in particular, vitamin B₁₂ supplementation and/or maintenance. Such a patient may be suffering from a vitamin B₁₂ deficiency, a disorder resulting from such a deficiency, or a disorder mimicking the symptomatology of such a deficiency due to, for example, malabsorption of vitamin B₁₂ as a result of the reduction in the digestive anatomy following bariatric surgery, e.g., reduced or insufficient secretion and/or utilization of intrinsic factor; dietary restrictions or changes as a result of compromised or failed kidney function, such as, for example, in a renal or dialysis patient; or recurring removal and loss of these vitamins from a renal patient's blood as a result of dialysis treatments. Examples of disorders resulting from or mimicking a vitamin B₁₂ deficiency include, for example, anemia, including pernicious anemia; nerve and brain degeneration, typically as a result of degradation or lack of myelin; and impaired heart or cardiovascular function.

The present treatment methods are also applicable for nutritional supplementation and maintenance of vitamin blood serum levels in a patient that is to undergo bariatric surgery (i.e., pre-operative supplementation and maintenance). Specifically, certain of the vitamins contained in the nutritional supplement disclose herein, and in particular vitamin B₁, are particularly beneficial for patients that are candidates for bariatric surgery. In particular, such candidates typically are in need of B₁, as it is essential for metabolism via its coenzyme functions. Specifically, such surgery candidates often have excess carbohydrate intake increasing the need for B₁. Because B₁ is not appreciably stored in the body, it may be depleted within two weeks or less on a B₁-free diet. Due to this, many bariatric surgical candidates may suffer from a B₁ deficiency even before surgery. Thus, in accordance with the present invention, the present nutritional supplement compositions are also particularly suitable for administration to patients that are candidates for bariatric surgery in accordance with the present methods providing for nutritional supplementation. The nutritional compositions disclosed herein may be orally administered to patients that are candidates for bariatric surgery in accordance with the methods disclosed herein with respect to bariatric surgery patients.

Similarly, the present treatment methods are also applicable for nutritional supplementation and maintenance of vitamin levels in a patient that is to undergo dialysis (i.e., pre-dialysis supplementation and maintenance). Specifically, the B-vitamins and vitamin C are removed from the blood during dialysis. Accordingly, proper loading of these vitamins in the body, and in particular the tissues, are particularly beneficial for these patients so that they have proper concentrations of the same ready for the body to use upon completion of the dialysis treatment. Thus, in accordance with the present invention, the present nutritional supplement compositions are also particularly suitable for administration to patients that are candidates for dialysis in accordance with the present methods providing for nutritional supplementation. The nutritional compositions disclosed herein may be orally administered to patients that are candidates for dialysis in accordance with the methods disclosed herein with respect to patients undergoing dialysis.

The present invention is directed particularly to the treatment of human patients having undergone bariatric surgery or that are renal or dialysis patients. However, it should be understood that the methods disclosed herein are generally applicable to the treatment of mammals including, for example, domesticated house pets, such as dogs and cats, as well as farm animals, such as cattle, pigs, horses, sheep and goats, wherein it is typically difficult to administer an otherwise non-dissolving tablet to the mammal, as methods of masking the tablet by the concurrent administration of liquids or, in particular, additional foodstuffs, makes the typical administration of such tablets difficult and sometimes physically impossible.

Methods for Nutritional Supplementation and Administration of Nutritional Composition

Generally, the supplementation methods of the present invention comprise the oral administration of a rapidly dissolvable tablet as described in greater detail herein.

Thus, one embodiment of the invention comprises orally administering to the patient a rapidly dissolving tablet, the tablet, in one embodiment, comprising an active ingredient component comprising a mixture of two or more B vitamins selected from the group consisting of B₁, B₂, B₃, B₅, B₆, B₇, B₉, and B₁₂; one or more non-B vitamins; and one or more minerals; a disintegrant; and a directly-compressible, water-soluble carbohydrate; wherein the tablet dissolves in the mouth of the patient within about 90 seconds or less after administration of the tablet and otherwise exhibits acceptable organoleptic properties. In another embodiment, the tablet comprises an active ingredient component comprising a mixture of two or more B vitamins selected from the group consisting of B₁, B₂, B₃, B₅, B₆, B₇, B₉, and B₁₂; one or more non-B vitamins; a disintegrant; and a directly-compressible, water-soluble carbohydrate; wherein the tablet dissolves in the mouth of the patient within about 90 seconds or less after administration of the tablet and otherwise exhibits acceptable organoleptic properties In a particular embodiment, the non-B vitamin is vitamin C. In accordance with a preferred embodiment, administration of the nutritional composition does not include concurrent oral administration of a liquid to the patient. However, it should be understood that the method of the present invention may include contemporaneous or concurrent administration of liquids to the patient in conjunction with administration of the nutritional supplement tablet.

Nutritional supplementation in accordance with the present invention comprises orally administering to the patient one or more rapidly dissolving tablets. In one embodiment, two such tablets, each containing the mixture of two or more B vitamins, one or more non-B vitamins, and optionally one or more minerals as detailed below, are orally administered. These tablets may be orally administered contemporaneously, such as for example, one or more tablets being administered simultaneously, or one or more tablets being administered within several minutes of one another (i.e., one tablet is administered, and, shortly after dissolving in the patient's mouth, a second tablet is orally administered). Alternatively, two or more tablets could be administered over a period of time, such as for example one tablet being orally administered at a given time of day and a second tablet being orally administered at a later time of day, such as for example, twelve hours later. In either instance, the purpose remains to provide nutritional supplementation to the patient in the form of an orally administered, rapidly dissolvable tablet that does not require the concurrent administration of a liquid.

The above described method may also comprise the step of administering an additional vitamin tablet that may or may not be rapidly dissolvable and that may or may not contain the same active ingredients or components as the rapidly dissolving tablet. In particular, as discussed in greater detail below, the rapidly dissolvable tablet may not contain therapeutic concentrations of calcium and/or magnesium. Therefore, it may be necessary to orally administer those minerals as a separate tablet. Accordingly, the present method for providing vitamin supplementation to a patient may also comprise the step of orally administering to the patient a second tablet, said tablet comprising an active ingredient component that is different from that contained in the rapidly dissolvable tablet. In this embodiment, typically the second tablet will be administered within about 4, preferably about 6, more preferably about 8, still more preferably about 10, and most preferably about 12 hours of administering the rapidly dissolvable tablet. In one embodiment, the second tablet is also a rapidly dissolving tablet, and more preferably a rapidly dissolving tablet comprising therapeutic concentrations of calcium and/or magnesium. The calcium and magnesium is preferably present in the citrate forms (i.e., as calcium citrate and magnesium citrate). In another embodiment, the second tablet is a hard, non-rapidly dissolving tablet (i.e., a tablet that is typically administered concurrently with at least a small amount of liquid to facilitate easy swallowing of the tablet), and more preferably a hard, non-rapidly dissolving tablet comprising therapeutic concentrations of calcium and/or magnesium.

In particular, with respect to candidates for bariatric surgery and candidates for dialysis, the methods disclosed above may be used to administer the nutritional supplements and/or additional vitamin tablets to a bariatric surgery candidate or dialysis candidate for at least about 8 weeks prior to surgery or the start of dialysis, more preferably for at least about 6 weeks prior to surgery or the start of dialysis, still more preferably for at least about 4 weeks prior to surgery or the start of dialysis, and most preferably for at least about 2 weeks prior to surgery or the start of dialysis. These same candidates, should they undergo surgery or dialysis, may be treated with the nutritional supplement compositions in accordance with the disclosed methods following surgery or dialysis.

Rapidly Dissolving Tablet—Active Ingredients

A further aspect of the present invention is a composition comprising one or more vitamins and/or minerals useful for treating nutritional deficiencies or maintaining vitamin blood serum levels in a bariatric surgery patient or a renal or dialysis patient. The composition may be used in previously known regimens for satisfying nutritional needs and treatment of nutritional deficiencies, but is specifically intended to be used in the practice of the treatment regimen of the present invention described above.

A composition of the present invention is generally a tablet (sometimes referred to as a troche or lozenge) containing the active and non-active (i.e., biologically inert) ingredient components (sometimes referred to as excipients) as detailed herein. In particular, the tablet is a rapidly dissolvable tablet containing these components, and in particular, in combination with a disintegrant or super disintegrant.

B Vitamins

In one embodiment, the rapidly dissolvable tablet comprises one or more B vitamins, and preferably two or more B vitamins. Typically, the B vitamins will be selected from the group consisting of B₁ (thiamine and thiamine HCl), B₂ (riboflavin), B₃ (niacin, niacinimid, nicotinic acid, and nicotinamide), B₅ (pantothenic acid and d-calcium pantothenate), B₆ (pyridoxine, pyridoxine HCl, pyridoxal, and pyridoxamine), B₇ (biotin, also known as Vitamin H), B₉ (folate and folic acid, also known as vitamin M), and B₁₂ (cobalamin). Any one or more of the B vitamins may be in any of the numerous forms of the vitamins known in the art, including pharmaceutically acceptable salts thereof, chemically modified equivalents thereof, and mixtures thereof. The selection of the specific form of any one or more of the B vitamins to be used in the composition depends upon a number of factors known to those of skill in the art, including, for example, the composition in which the vitamins are to be mixed or dissolved, the amount or concentration of the vitamins desired in the composition, the solubility of the vitamins, and the pH of the composition or resulting tablet. With respect to vitamin B₁₂ in particular, the cobalamin may be any of a number of known cobalamin compounds, including for example, cyanocobalamin, hydroxocobalamin (vitamin B₁₂ₐ), hydroxocobalamin HCl, sulfate, acetate and other hydroxocobalamin salts, aquacobalamin (vitamin B₁₂), nitrilocobalamin (vitamin B_{12c}), methylcobalamin (methyl B₁₂), 5′-deoxyadenosine cobalamin (coenzyme B₁₂), pharmaceutically acceptable salts thereof, chemically modified equivalents thereof, and mixtures thereof. Preferably, the cobalamin is cyanocobalamin.

Each of the B vitamins is beneficial to human health for various reasons, having differing absorption and toxicity issues. B₁ (also commonly referred to as thiamin(e), thiamine HCl, and thiamine mononitrate) is, for example, important for functions critical for energy production and nerve transmission. A common disorder resulting from vitamin B₁ deficiency is termed beriberi. Symptoms of this disorder range from vague fatigue, weakness, and confusion to difficulty walking up stairs, standing on one leg, and hallucination or psychosis. Vitamin B₁ is typically easily absorbed by the body; however, surgical bypass of the upper small intestine eliminates some of the primary absorption sites for it. In addition, ethanol is known to block the transport of B₁ from the gut into the body. Magnesium is useful for the conversion of B₁ to its active form, and, therefore, magnesium deficiency can contribute to thiamine deficiency. B₁ is water soluble and has no listed tolerable upper level (UL) because no evidence of oral toxicity has been seen even with doses as high as 200 mg taken for extended periods of time. Generally, vitamin B₁, if present, will be in an amount of about 0.375 mg to about 100 mg per tablet, preferably about 1 mg to about 50 mg per tablet, more preferably about 1.35 mg per tablet to about 2.25 mg per tablet, and most preferably in an amount of about 1.5 mg per tablet. Notably, the amount of B₁ present in a nutritional supplement tablet when utilized in surgical candidates may be significantly higher than it is in a tablet typically used in a post-surgery patient. In such an instance, B₁ will be in a nutritional supplement for bariatric patients in an amount of about 50 mg to about 100 mg per tablet, preferably about 75 mg to about 100 mg per tablet, more preferably about 75 mg per tablet, and most preferably in an amount of about 100 mg per tablet. The amount of B₁ present in a nutritional supplement for renal and dialysis patients will generally be in an amount of about 0.75 mg to about 200 mg per tablet, preferably about 0.75 mg to about 100 mg per tablet, more preferably about 1 mg to about 50 mg per tablet, still more preferably about 1 mg to about 25 mg per tablet, even more preferably in an amount of about 1.5 mg per tablet, and most preferably in an amount of about 2.0 mg per tablet.

B₂ (also commonly referred to as riboflavion) is, for example, important for conversion of carbohydrates into sugar. Symptoms of B₂ deficiency include fatigue, slowed growth, digestive problems, cracks and sores around the corners of the mouth, eye fatigue, and sensitivity to light. B₂ is typically readily absorbed from the upper gastrointestinal (GI) tract; however, absorption involves active transport mechanisms and the extent of GI absorption may be limited by the duration of contact of the drug with the specialized segment of mucosa where absorption occurs. The extent of GI absorption of B₂ may be increased when the nutritional supplement is administered with food and may be decreased in patients with hepatitis, cirrhosis, or biliary obstruction. B₂ is a water soluble nutrient for which no tolerable upper level has been established. Generally, vitamin B₂, if present, in a nutritional supplement for bariatric patients will be in an amount of about 0.45 mg to about 100 mg per tablet, preferably about 0.45 mg to about 2.25 mg per tablet, more preferably about 0.85 mg per tablet to about 1.28 mg per tablet, and most preferably in an amount of about 0.85 mg per tablet. The amount of B₂, if present, in a nutritional supplement for renal and dialysis patients will generally be in an amount of about 0.85 mg to about 200 mg per tablet, preferably about 0.85 mg to about 100 mg per tablet, more preferably about 1 mg to about 50 mg per tablet, still more preferably about 1 mg to about 25 mg per tablet, even more preferably in an amount of about 1.7 mg per tablet, and most preferably in an amount of about 2.3 mg per tablet.

B₃ (also commonly referred to as niacin and niacinamide), for example, converts carbohydrates into glucose and plays and important role in maintaining muscle tone along the digestive tract, as well as promoting the health of the nervous system, skin, hair, eyes, mouth, and liver. Symptoms of a B₃ deficiency include, for example, indigestion, fatigue, canker sores, vomiting, and depression. Both acid and amide forms are readily absorbed in the small intestine. Toxicity is possible; the Institute of Medicine lists 35 mg/day as the tolerable upper limit for niacin. Generally, vitamin B₃, if present, in a nutritional supplement for bariatric patients will be in an amount of about 5 mg to about 50 mg per tablet, preferably about 5 mg per tablet to about 17.5 mg per tablet, more preferably about 9 mg per tablet to about 15 mg per tablet, and most preferably in an amount of about 10 mg per tablet. The amount of B₃, if present, in a nutritional supplement for renal and dialysis patients will generally be in an amount of about 5 mg to about 100 mg per tablet, preferably about 5 mg to about 50 mg per tablet, more preferably about 10 mg to about 30 mg per tablet, still more preferably about 20 mg to about 30 mg per tablet, even more preferably in an amount of about 20 mg per tablet, and most preferably in an amount of about 27 mg per tablet.

B₅ (also commonly referred to as pantothenoic acid), for example, is a constituent of coenzyme A. It plays an important role in the metabolism of carbohydrates, proteins, and fats and is, therefore, important for the repair of all cells and tissues. It is readily absorbed in the small intestine. B₅ is a water soluble nutrient for which the Institute of Medicine does not list a tolerable upper level. Generally, vitamin B₅, if present, in a nutritional supplement for bariatric patients will be in an amount of about 2.5 mg to about 15 mg per tablet, preferably about 4.5 mg per tablet to about 7.5 mg per tablet, and most preferably in an amount of about 5 mg per tablet. The amount of B₅, if present, in a nutritional supplement for renal and dialysis patients will generally be in an amount of about 1 mg to about 30 mg per tablet, preferably about 1 mg to about 20 mg per tablet, more preferably about 1 mg to about 10 mg per tablet, still more preferably about 2.5 mg to about 10 mg per tablet, even more preferably in an amount of about 5 mg per tablet, and most preferably in an amount of about 6.8 mg per tablet.

B₆ (also commonly referred to as pyridoxine HCL) serves, for example, as a coenzyme and is involved in the metabolism of protein and carbohydrates, the production of insulin and blood cells, and the synthesis of neurotransmitters, enzymes, and prostoglandins. Symptoms of B₆ deficiency are vaguely defined and include, for example insomnia, irritability, nervousness, muscle weakness, and difficulty in walking, and may result in fissures and cracking at the corners of the mouth. B₆ is typically readily absorbed in the small intestine. Excess vitamin B is excreted in the urine so adequate daily intake is essential. B₆ intake at high concentrations for extended periods of time can be toxic. For example, the Institute of Medicine tolerable upper limit is 100 mg/day. A long term study utilizing administration of 200 mg/day of B₆ demonstrated potential damage to sensory nerves. Generally, vitamin B₆, if present, in a nutritional supplement for bariatric patients will be in an amount of about 0.5 mg to about 50 mg per tablet, preferably about 2.25 mg per tablet to about 5 mg per tablet, more preferably about 2.25 mg per tablet to about 3.75 mg per tablet, and most preferably in an amount of about 2.5 mg per tablet. The amount of B₆, if present, in a nutritional supplement for renal and dialysis patients will generally be in an amount of about 1 mg to about 100 mg per tablet, preferably about 1 mg to about 50 mg per tablet, more preferably about 5 mg to about 25 mg per tablet, still more preferably about 10 mg to about 20 mg per tablet, even more preferably in an amount of about 10 mg per tablet, and most preferably in an amount of about 14 mg per tablet.

B₇ (also commonly referred to as biotin), for example, plays a role in carbohydrate, fat, and protein metabolism and is necessary for normal growth and body function. It is readily absorbed in the small intestine, and in particular in the upper part of the small intestine. B₇ is a water soluble nutrient for which the Institute of Medicine has no published tolerable upper level. Generally, vitamin B₇, if present, in a nutritional supplement for bariatric patients will be in an amount of about 22.5 μg to about 450 μg per tablet, preferably about 135 μg per tablet to about 225 μg per tablet, and most preferably in an amount of about 150 μg per tablet. The amount of B₇, if present, in a nutritional supplement for renal and dialysis patients will generally be in an amount of about 0.001 mg to about 450 mg per tablet, preferably about 0.01 mg to about 100 mg per tablet, more preferably about 0.1 mg to about 50 mg per tablet, still more preferably about 0.3 mg to about 10 mg per tablet, even more preferably in an amount of about 0.3 mg per tablet, and most preferably in an amount of about 0.4 mg per tablet.

B₉ (also commonly referred to as folate or folic acid), for example, is involved in the synthesis of nucleic acids and the synthesis of methionine from homocysteine. B₉ deficiencies can affect cell growth, leading to specific forms of cancer and birth defects. B₉ in food must be hydrolyzed, and the best B₉ absorption occurs on an empty stomach. B₉ is primarily absorbed in the jejunum, with absorption also occurring in the duodenum. Notably, a zinc deficiency can contribute to B₉ deficiency. The Institute of Medicine lists 1000 μg as the tolerable upper limit for folic acid and limits over the counter supplements to 800 μg. This is not, however, because of toxicity; rather, it is because high-dose folate can mask B₁₂ deficiency. In the absence of a patient's B₁₂ deficiency, B₉ is safe at almost any dose. Generally, vitamin B₉, if present, in a nutritional supplement for bariatric patients will be in an amount of about 100 μg to about 1000 μg per tablet, preferably about 270 μg per tablet to about 450 μg per tablet, and most preferably in an amount of about 300 μg per tablet. It should be understood that in accordance with the present invention, higher dosages of B₉ could be present in a particular embodiment of the tablet designated for patients also taking supplemental B₁₂ (e.g., nasally administered B₁₂) under the supervision of medical personnel. In particular, such personnel could act to mitigate the risk associated with masking of the B₁₂ deficiency by B₉, this masking effect being the primary reason for limiting the amount of B₉ in the disclosed nutritional supplement tablet. The amount of B₉, if present, in a nutritional supplement for renal and dialysis patients will generally be in an amount of about 0.1 mg to about 20 mg per tablet, preferably about 0.1 mg to about 10 mg per tablet, more preferably about 1 mg to about 10 mg per tablet, still more preferably about 2 mg to about 5 mg per tablet, even more preferably in an amount of about 2 mg per tablet, and most preferably in an amount of about 2.7 mg per tablet.

B₁₂ (also commonly referred to as cobalamin), for example, is required for the creation of energy from dietary fats and proteins, the production of hemoglobin, the reduction of homocysteine back to methionine, and the synthesis of s-adenosyl-methionine (SAM-e). A B₁₂ deficiency can result in a toxic build-up of homocysteine. Absorption of B₁₂ takes place in the small intestine and generally requires the presence of intrinsic factor (IF), a protein found in the stomach. B₁₂ ingested in food is generally bound by IF, the resultant complex typically reaching the intestine within 2-3 hours thereafter. There is no known tolerable upper limit for B₁₂; toxicity has not been demonstrated, even at levels 10,000× the recommended daily allowance (RDA). Generally, vitamin B₁₂, if present, in a nutritional supplement for bariatric patients will be in an amount of about 1.5 μg to about 375 μg per tablet, preferably about 110 μg per tablet to about 190 μg per tablet, and most preferably in an amount of about 125 μg per tablet. The amount of B₁₂, if present, in a nutritional supplement for renal and dialysis patients will generally be in an amount of about 0.001 mg to about 100 mg per tablet, preferably about 0.002 mg to about 50 mg per tablet, more preferably about 0.005 mg to about 25 mg per tablet, still more preferably about 0.006 mg to about 10 mg per tablet, even more preferably in an amount of about 0.006 mg per tablet, and most preferably in an amount of about 0.0081 mg per tablet.

Non-B Vitamins

The rapidly dissolvable tablet typically comprises one or more non-B vitamins as well. Those vitamins may be any such non-B vitamin, including both fat-soluble and water-soluble vitamins, but typically will be one or more vitamins selected from the group consisting of vitamin A, vitamin C, vitamin D, vitamin E, and vitamin K. Preferably, the tablet will comprise the non-B vitamins A, C, D, and E. In a particular embodiment, the non-B vitamin is vitamin C. Any one or more of the non-B vitamins may be in any of the numerous forms of the vitamins known in the art, such as, for example, acetate and beta-carotene (vitamin A), ascorbic acid (vitamin C), vitamin D₃/cholecalciferol and ergocalciferol (vitamin D), and d-alpha tocopheryl succinate (vitamin E), and includes pharmaceutically acceptable salts thereof, chemically modified equivalents thereof, and mixtures thereof. The selection of the specific form of any one or more of the non-B vitamins to be used in the composition depends upon a number of factors known to those of skill in the art, including, for example, the composition in which the vitamins are to be mixed or dissolved, the amount or concentration of the vitamins desired in the composition, the solubility of the vitamins, and the pH of the composition or resulting tablet.

Vitamin A (also commonly referred to as vitamin A acetate, retinyl palmitate, retinyl acetate, or its provitamin equivalent β-carotene & other carotenoids) is, for example, important to eye health, immunity, transport of iron into the hemoglobin of developing red blood cells, and hormonal actions. Vitamin A deficiency can cause blindness, skin irritation, infection, and poor wound healing. Retinol absorption of vitamin A tends to be much more efficient than carotenoid absorption. Retinal formed from retinol via zinc is critical to night vision; therefore, low zinc status can decrease night vision without the presence of a true vitamin A deficiency. Because vitamin A plays a role in the transport of iron into hemoglobin and red blood cell formation, a vitamin A deficiency can contribute to anemia. The Institute of Medicine lists 3000 μg/day (10,000 IU/day) as the tolerable upper limit. Generally, vitamin A, if present, will be in an amount of about 875 IU per tablet to about 7300 IU per tablet as preformed vitamin A, pro vitamin A, or a mixture thereof, preferably about 2250 IU per tablet to about 4125 IU per tablet as preformed vitamin A, pro vitamin A, or a mixture thereof, and most preferably in an amount of about 2500 IU per tablet as preformed vitamin A, pro vitamin A, or a mixture thereof.

Vitamin C (also commonly referred to as ascorbic acid or sodium ascorbate) is, for example, important for the manufacture of collagen, wound healing, a healthy immune and nervous system, adrenal hormone production, and as an antioxidant to help prevent disease. Absorption generally occurs in the intestine. The Institute of Medicine lists 2000 mg/day as the tolerable upper limit. Generally, vitamin C, if present, will be in an amount of about 15 μg to about 1000 μg per tablet, preferably about 15 μg per tablet to about 120 μg per tablet, more preferably about 27 μg per tablet to about 45 μg per tablet, and most preferably in an amount of about 30 μg per tablet. In certain embodiments, the vitamin C is present both in the form of ascorbic acid and sodium ascorbate. In such an embodiment, the ascorbic acid may generally be present in an amount of about 1 mg to about 100 mg per tablet, preferably about 1 mg to about 50 mg per tablet, more preferably about 5 mg to about 25 mg per tablet, still more preferably about 10 mg to about 20 mg per tablet, even more preferably in an amount of about 18 mg per tablet, and most preferably in an amount of about 24 mg per tablet, while the sodium ascorbate may generally be present in an amount of about 1 mg to about 100 mg per tablet, preferably about 5 mg to about 75 mg per tablet, more preferably about 10 mg to about 50 mg per tablet, still more preferably about 35 mg to about 50 mg per tablet, even more preferably in an amount of about 42 mg per tablet, and most preferably in an amount of about 57 mg per tablet.

Vitamin D (also commonly referred to as cholecalciferol or ergocalciferol) is, for example, important for the absorption of calcium for bone health. In addition, it may play a role in immunity, diabetes, and heart health. Absorption of dietary vitamin D takes place in the upper part of the small intestine, generally with the aid of bile salts. D₃ (cholecalciferol) is considered the most potent form. The Institute of Medicine lists 2000 IU/day as the tolerable upper limit. Generally, vitamin D, if present, will be in an amount of about 100 IU per tablet to about 1000 IU per tablet, preferably about 100 IU per tablet to about 660 IU per tablet, more preferably about 360 IU per tablet to about 660 IU per tablet, and most preferably in an amount of about 400 IU per tablet.

Vitamin E (also commonly referred to as d-Alfa tocopheryl acid succinate (natural form), dl-alpha tocopheryl acid succinate (synthetic form—only d active), and tocotrienols (less active form)) is an antioxidant that, for example, aids in the prevention or inhibition of neuromuscular impairment. Vitamin E is readily absorbed, with the natural and d-alpha tocopherol forms being absorbed better than the synthetic dl-alpha tocopheryl acid succinate form. The Institute of Medicine lists 1500 IU/day as the tolerable upper limit. Impaired thyroid function can result from the long term intake of 600 IU/day. Generally, vitamin E, if present, will be in an amount of about 7.5 IU per tablet to about 750 IU per tablet, preferably about 15 IU per tablet to about 300 IU per tablet, more preferably about 13.5 IU per tablet to about 24.75 IU per tablet, and most preferably in an amount of about 15 IU per tablet.

Additional non-B vitamins, such as for example, vitamin K, may also be present in the rapidly dissolving tablets disclosed herein. Concentrations and forms of such vitamins for such a tablet are readily determined by one of ordinary skill in the art according to factors well known in the medical arts.

Minerals

The rapidly dissolvable tablet typically, although not necessarily, comprises one or more minerals as well, particularly when administered as a composition for providing nutritional supplementation to bariatric patients. Those minerals may include any minerals generally known in the art to be dietary minerals which are beneficial to human health, and in particular, those often referred to as essential minerals which are necessary for ideal human health. Examples of minerals that may be present in the tablet include, for example, boron, bromine, cadmium, calcium, chloride, chromium, cobalt, copper, fluoride, iodine, iron, magnesium, manganese, molybdenum, nickel, phosphorous, potassium, selenium, silicon, sodium, tin, tungsten, vanadium, and zinc. Preferably, the tablet will comprise one or more minerals selected from the group consisting of chromium, copper, iron, manganese, molybdenum, selenium, and zinc. Any one or more of the minerals may be in any of the numerous forms known in the art. By way of example, minerals may be in the form of an amino acid chelate with respect to chromium, molybdenum, and selenium, a citrate with respect to copper, manganese, and zinc, and a fumarate with respect to iron, and includes pharmaceutically acceptable salts thereof, chemically modified equivalents thereof, and mixtures thereof. The selection of the specific form of any one or more of the minerals to be used in the composition depends upon a number of factors known to those of skill in the art, including, for example, the composition in which the minerals are to be mixed or dissolved, the amount or concentration of the minerals desired in the composition, the solubility of the minerals, and the pH of the composition or resulting tablet.

In one preferred embodiment, the tablet of the present invention comprises the minerals chromium, copper, iron, manganese, molybdenum, selenium, and zinc, and is free of therapeutic amounts of calcium and/or magnesium. It has been determined that the exclusion of therapeutic quantities of calcium and/or magnesium reduces tablet size, and optimizes dissolution rate, taste, and mouth feel of the tablet. More particularly, exclusion of therapeutic quantities of calcium and/or magnesium allows inclusion of sufficient quantities of non-active ingredients (e.g., directly compressible mannitol) necessary to provide a rapidly dissolvable tablet. The exclusion of therapeutic amounts of calcium from the rapidly dissolvable tablet, and oral administration of the same in a second, separate tablet, may also enhance overall calcium absorption, as the iron in the rapidly dissolvable tablet can potentially interfere with calcium absorption if both ingredients are administered in the same tablet. It should be understood that in this preferred embodiment free of therapeutic amounts of calcium and magnesium, sub-therapeutic amounts of calcium and/or magnesium may be present, for example, as a cation associated with other ingredients in salt form, such as, for example, magnesium stearate used as a lubricant or calcium pantothenate.

In another preferred embodiment, particularly useful for providing nutritional supplementation to renal and dialysis patients, the tablet of the present invention is free of therapeutic quantities of phosphorous, potassium, and/or sodium, and more preferably essentially free of phosphorous, potassium, and/or sodium. Because renal and dialysis patients have compromised or failed kidney function, concentrations of these minerals in the blood as a result of consumption of normal dietary amounts of the same would result in excess blood serum concentrations of these minerals. Because the excess amounts can only be removed during dialysis treatments (as opposed to continually when kidney function is normal), serious health risks and dietary complications (i.e., increased thirst, resulting in increased fluid intake which is also severely restricted in the diet of renal and dialysis patients) can result. Thus, patients already on a diet restricting the intake of phosphorous, potassium, and/or sodium, as well as fluids, need nutritional supplementation that is substantially or completely free of these minerals.

Chromium (also commonly referred to as chromium chelate), for example, aids transport of glucose into cells and enhances the effect of insulin in glucose utilization. A chromium deficiency can result in reduced tissue sensitivity to glucose similar to that seen patients suffering from diabetes, numbness in extremities, and disturbances of glucose, fat, and protein metabolism. Chromium is readily absorbed, with organic chromium being absorbed more efficiently than inorganic chromium. There is little known about chromium toxicity, although there is some evidence that doses above 250 μg can cause adverse reactions. Dietary forms of the mineral, however, have very low toxicity. Generally, chromium, if present, will be in an amount of about 30 μg to about 125 μg per tablet, preferably about 30 μg per tablet to about 60 μg per tablet, more preferably about 42 μg per tablet to about 78 μg per tablet, and most preferably in an amount of about 60 μg per tablet.

Copper (also commonly referred to as copper citrate, cupric oxide, copper amino acid chelate, copper gluconate, and copper sulfate) is, for example, important for normal metabolism; healthy bones, joints, skin, and blood vessels; a healthy nervous, cardiovascular, and immune systems; the formation of hemoglobin in red blood cells; and antioxidant action. It is absorbed in the stomach and small intestine. The Institute of Medicine lists 10 mg/day as the tolerable upper limit. Generally, copper, if present, will be in an amount of about 0.5 mg to about 5 mg per tablet, preferably about 0.7 mg per tablet to about 2 mg per tablet, more preferably about 0.7 mg per tablet to about 1.3 mg per tablet, and most preferably in an amount of about 1 mg per tablet. In a preferred embodiment, copper citrate, as opposed to the chelate form, is used as the copper source to improve the taste of the tablet.

Iron (also commonly referred to as ferrous (Fe²⁺) fumarate and carbonyl iron) is, for example, important for the storage of oxygen in red blood cells and muscle cells, immunity, growth, development, and tissue repair. An iron deficiency can result in anemia, hair loss, shortness of breath, and a swollen tongue. The Institute of Medicine lists 45 mg/day as the tolerable upper limit. Acute toxicity can occur at intake levels of 40 mg/Kg, and a single dose 200+ mg/Kg can be fatal. Generally, iron, if present, will be in an amount of about 2.8 mg to about 18 mg per tablet, preferably about 2.8 mg per tablet to about 9 mg per tablet, more preferably about 2.8 mg per tablet to about 5.2 mg per tablet, and most preferably in an amount of about 4 mg per tablet. The amount of iron may also be increased to an amount of about 9 mg per tablet in order to achieve the FDA RDI of 18 mg per day. This may be accomplished utilizing the forms of iron disclosed herein. Alternatively, because iron has the potential to impart a less than pleasant taste to the tablet and can also lead to other minor side effects such as stomach irritation, increasing the amount of iron to a still more preferred amount of about 9 mg per tablet may be more readily achieved if the form of iron used is well suited to this type of tablet, such as, for example, forms of iron with very little taste or that are gentler on the stomach.

Manganese (also commonly referred to as manganese citrate, manganese amino acid chelates, manganese sulfate, and manganese gluconate) is, for example, important for energy production, the action of the antioxidant enzyme superoxide dismutase, protein metabolism, bone formation, and a healthy nervous system. Typically, only 3-5% of dietary manganese is absorbed. The Institute of Medicine lists 11 mg/day as the tolerable upper limit. Generally, manganese, if present, will be in an amount of about 0.5 mg to about 5.5 mg per tablet, preferably about 0.7 mg per tablet to about 2 mg per tablet, more preferably about 0.7 mg per tablet to about 1.3 mg per tablet, and most preferably in an amount of about 1 mg per tablet.

Molybdenum (also commonly referred to as molybdenum chelate, sodium molybdate) is, for example, important for reactions involving the waste products of protein metabolism, iron utilization, carbohydrate metabolism, and alcohol and sulfite detoxification. Deficiency is rare, as molybdenum is readily absorbed from dietary intake. Molybdenum, does however, compete with copper at absorption sites and, therefore, can inhibit copper absorption at higher doses. The Institute of Medicine lists 2000 μg/day as the tolerable upper limit; however, concentrations above 500 μg/day have been found to cause significant losses of copper. Generally, molybdenum, if present, will be in an amount of about 11.25 μg to about 1000 μg per tablet, preferably about 22.5 μg per tablet to about 150 μg per tablet, more preferably about 26.25 μg per tablet to about 48.75 μg per tablet, and most preferably in an amount of about 37.5 μg per tablet.

Selenium (also commonly referred to as selenium Citrate, sodium selenite, and l-selenomethionine) is, for example, important to thyroid activity and may play an important role in immune function and cancer prevention. Organic selenium is absorbed more efficiently than inorganic forms. The Institute of Medicine lists 400 μg/day as the tolerable upper limit; however, a study of healthy men fed diets with approximately 300 μg of selenium developed clinical hypothyroidism and weight gain after only 99 days. Generally, selenium, if present, will be in an amount of about 13 μg to about 200 μg per tablet, preferably about 13 μg per tablet to about 150 μg per tablet, more preferably about 19.25 μg per tablet to about 35.75 μg per tablet, and most preferably in an amount of about 27.5 μg per tablet.

Zinc (also commonly referred to as zinc citrate, zinc oxide, zinc methionine, zinc gluconate, zinc sulfate, zinc picolinate, and chelated zinc) is, for example, important for growth and development, skin health, host defense, immunity, protein & cell membrane structure, and genetic transcription. In addition zinc-dependant enzymes catalyze digestion, immune function, and antioxidant activity. Zinc deficiency symptoms include loss of smell, abnormal or diminished sense of taste, poor wound healing, skin rashes, and thinning hair. Zinc is readily absorbed by the body, with organic forms of zinc being better absorbed than other forms. The Institute of Medicine lists 40 mg/day as the tolerable upper limit. Acute toxicity can occur at doses as low as 30 mg/day, but is more common with doses exceeding 100 mg/day. Generally, zinc, if present, will be in an amount of about 3.75 mg to about 20 mg per tablet, preferably about 3.75 mg per tablet to about 15 mg per tablet, more preferably about 5.25 mg per tablet to about 9.75 mg per tablet, and most preferably in an amount of about 7.5 mg per tablet. In a particular embodiment, zinc is present in an amount of about 15 mg, and in another embodiment in an amount of about 20.25 mg. In a preferred embodiment, zinc citrate, as opposed to the chelate form, is used as the zinc source to improve the taste of the tablet.

Additional minerals, such as for example, boron, bromine, cadmium, calcium, chloride, cobalt, fluoride, iodine, magnesium, nickel, phosphorous, potassium, silicon, sodium, tin, tungsten, and vanadium may also be present in the rapidly dissolving tablets disclosed herein. Concentrations and forms of each of these minerals for such a tablet are readily determined by one of ordinary skill in the art according to factors well known in the medical arts. In particular, forms of the rapidly dissolvable tablet intended for providing nutritional supplementation to renal or dialysis patients will generally be free of phosphorous, potassium, and/or sodium.

In another embodiment, therapeutic quantities of magnesium, calcium, or a combination of magnesium and calcium are also included in the tablet. Generally, magnesium, if present in a therapeutic quantity, is present as magnesium citrate in an amount of about 12.5 mg to about 400 mg per tablet, preferably about 12.5 mg to about 200 mg per tablet, more preferably about 12.5 mg per tablet to about 25 mg per tablet, and most preferably in an amount of about 25 mg per tablet. Generally, calcium, if present in a therapeutic quantity, is present as calcium citrate in an amount of about 25 mg to about 1000 mg per tablet, preferably about 200 mg per tablet to about 600 mg per tablet, more preferably about 500 mg per tablet to about 600 mg per tablet, and most preferably in an amount of about 600 mg per tablet.

Other Nutrients

Additional nutrients, such as, for example, choline, inositol, bioflavinoids (such as citrus fruit peel bioflavinoids), arabinogalactans, probiotics (such as lactobacillus acidophilus), ginseng, ginko, lutein, lycopene, phytosterols, and other nutrients commonly found in multi-vitamins, may also be present in the rapidly dissolving tablets disclosed herein. Concentrations and forms of such nutrients for such a tablet are readily determined by one of ordinary skill in the art according to factors well known in the medical arts.

Many of the ingredient components, and in particular, the active ingredient components, of the present tablets are listed as a per tablet amount, with the dose of the composition being two tablets. It is to be understood that the present ingredient components could also be formulated such that a dose could be administered as a single tablet, or as more than two tablets. The recitation of the ingredient components on a per tablet basis, with a dose being two tablets, is merely to be construed as exemplary, and in certain circumstances a preferred embodiment, of the nutritional supplement compositions disclosed herein.

In a particular embodiment, the active ingredient component of the rapidly dissolvable tablet is particularly suited for providing nutritional supplementation to a bariatric patient or candidate and has the formulation disclosed in Table 1.

TABLE 1
Specific active ingredient component formulation of
rapidly dissolving tablet for bariatric patients

| | | Daily | | | |
|---|---|---|---|---|---|
| | | Dose 2- | | Unit of | |
| Nutrient | Source | Tab | RDI/DRI | measure | % DV |
| Vitamin B₁ | Thiamine HCl | 3 | 1.5 | mg | 200 |
| Vitamin B₂ | Riboflavin | 1.7 | 1.7 | mg | 100 |
| Vitamin B₆ | Pyridoxine HCl | 5 | 2 | mg | 250 |
| Vitamin B₁₂ | Cyanocobalamin | 250 | 6 | μg | 4167 |
| Folic Acid | Folic Acid | 0.6 | 0.4 | mg | 150 |
| Niacinamide | Niacinamide | 20 | 20 | mg | 100 |
| Pantothenoic Acid | d-calcium pantothenate | 10 | 10 | mg | 100 |
| Biotin | Biotin | 300 | 300 | μg | 100 |
| Vitamin C | Ascorbic Acid | 60 | 60 | mg | 100 |
| Vitamin A | Acetate | 5000 | 5000 | IU | 100 |
| Vitamin D | Cholecalciferol | 800 | 400 | IU | 200 |
| Vitamin E | d-alfa tocopheryl acid | 30 | 30 | IU | 100 |
| | succinate | | | | |
| Iron | Ferrous fumarate | 8 | 18 | mg | 44 |
| Selenium | Amino Acid Chelate | 55 | 55 | μg | 100 |
| Copper | Citrate | 2 | 2 | mg | 100 |
| Manganese | Citrate | 2 | 2 | mg | 100 |
| Chromium | Amino Acid Chelate | 120 | 120 | μg | 100 |
| Molybdenum | Amino Acid Chelate | 75 | 75 | μg | 100 |
| Zinc | Citrate | 15 | 15 | mg | 100 |

In another embodiment, the active ingredient component of the rapidly dissolvable tablet is particularly suited for providing nutritional supplementation to a renal or dialysis patient or candidate and has the formulation disclosed in Table 2.

TABLE 2
Specific active ingredient component formulation of rapidly
dissolving tablet for renal or dialysis patents

| | | Daily | | | |
|---|---|---|---|---|---|
| | | Dose 2- | | Unit of | |
| Nutrient | Source | Tab | RDI/DRI | measure | % DV |
| Vitamin B₁ | Thiamine HCl | 0.3 | 1.5 | mg | 20 |
| Vitamin B₂ | Riboflavin | 1.7 | 1.7 | mg | 100 |
| Vitamin B₆ | Pyridoxine HCl | 10 | 2 | mg | 500 |
| Vitamin B₁₂ | Cyanocobalamin | 6 | 6 | μg | 100 |
| Folic Acid | Folic Acid | 2 | 0.4 | mg | 500 |
| Niacinamide | Niacinamide | 20 | 20 | mg | 100 |
| Pantothenoic Acid | calcium pantothenate | 5 | 10 | mg | 50 |
| Biotin | D-Biotin | 300 | 300 | μg | 100 |
| Vitamin C | Ascorbic Acid | 18 | 60 | mg | 30 |
| Vitamin C | Sodium ascorbate | 42 | 60 | mg | 70 |
| Zinc (optional) | Zinc citrate | 15 | 15 | mg | 100 |

In a preferred embodiment, the composition may be sugar free. Sugar free is generally understood to mean that the tablet or daily dose (e.g., two tablets) contains less than 0.5 g of sugars per reference amount customarily consumed and per label serving (e.g., one or two tablets). Sugar free as used herein is understood to include a nutritional supplement composition that contains a sugar alcohol (also known as a polyol, a polyhydric alcohol, or a polyalcohol), such as, for example, glycol, glycerol, erythritol, xylitol, mannitol, sorbitol, malitol, and lactitol, and other compounds having the general formula H(HCHO)ₙ₊1H (as opposed to the formula H(HCHO)ₙH. of the related sugar) and which generally do not result in a typically change in blood glucose levels associated with consumption of a sugar.

Rapidly Dissolving Tablet—Non-Active Ingredients

In addition to the active ingredient components, the rapidly dissolvable tablets of the present invention also contain non-active ingredients (sometimes referred to herein as “non-active components” or as “excipients”) that allow for the active ingredients to be formulated as a tablet that rapidly dissolves in the mouth of a patient upon oral administration. Such ingredients include, for example, one or more of a diluent or filler, a binder, a disintegrant, a lubricant, a sweetener, and a flavoring agent. Moreover, it should be understood that certain non-active ingredients may have multiple functions, such as filler, binder, and disintegrant or binder, filler, and sweetener.

In one embodiment, the rapidly dissolvable tablet of the present invention comprises a rapidly dissolvable sugar or sugar alcohol that serves as a binder and filler. This sugar or sugar alcohol may also serve as a sweetener, aiding in masking the otherwise unpleasant tastes of the active ingredient component contained in the tablet. Examples of such sugars or sugar alcohols include, for example, mannitol, lactose, sucrose, maltose, dextrose, sorbitol, xylitol, maltitol, lactitol, and maltodextrins. Mannitol and other similar compounds having a negative heat of solution are preferred because they provide a particularly pleasant sensation enhancing the organoleptic experience of ingesting the described tablet. Preferably, the rapidly dissolvable sugar or sugar alcohol binder and filler (sometimes referred to herein as a “binder/filler”) is a directly-compressible, water-soluble carbohydrate. For example, directly compressible mannitol may be used. An example of a product containing directly compressible mannitol is LUDIFLASH® (BASF, Switzerland), and in one preferred embodiment, the rapidly dissolvable sugar or sugar alcohol binder and filler is a directly-compressible, water-soluble carbohydrate, particularly mannitol, and more particularly mannitol present in LUDIFLASH®. In accordance with another preferred embodiment, the directly compressible mannitol is Roquette's PEARLITOL® 100 SD as described in U.S. Pat. No. 5,573,777, the content of which is hereby incorporated herein by reference.

The tablet in accordance with the present invention generally comprises between about 5% and about 80% of the rapidly dissolvable sugar or sugar alcohol binder and filler based on the weight of the tablet. More preferably, the amount of such filler will range from about 50% to about 80% by weight based on the tablet, still more preferably from about 70% to about 80% by weight, and most preferably from about 75% to about 80% by weight of the tablet.

The rapidly dissolvable tablet of the present invention also preferably comprises a disintegrant or a super disintegrant to aid in the rapid dissolution of the tablet in the mouth of a patient upon oral administration of the tablet. Classical disintegrants include, for example, starches, pre-gelatinized starches, alginates, low-substituted hydroxypropylcellulose, and microcrystalline cellulose. Super disintegrants include, for example, sodium starch glycolate (an example of a crosslinked starch), croscarmellose and croscarmellose sodium (also known as crosslinked sodium carboxymethyl cellulose) (examples of a crosslinked cellulose), crospovidone (polyvinyl pyrrolidone (“PVP”)) (an example of a crosslinked polymer), gellan gum, and Xanthan SM, and are generally water insoluble. While they add to the rapid disintegration of the formulation, their inclusion can also add to the total content of insoluble ingredients making it more difficult to strike a balance between disintegration/dissolution speed and the resulting organoleptic sensation. Accordingly, the amount of disintegrant or super disintegrant will range from about 0.5% to about 10% by weight based on the weight of the tablet, preferably from about 1% to about 10% by weight based on the weight of the tablet, and more preferably from about 1% to about 5%. An amount of disintegrant or super disintegrant that is 5% or less by weight of the tablet is most preferred. While polyvinyl pyrrolidone, a composition comprising polyvinyl pyrrolidone, croscarmellose, croscarmellose sodium, sodium starch glycolate, gellan gum, or Xanthan SM, a combination of any one of these compounds, or a composition comprising any one or more of these compounds are the preferred super disintegrants, with polyvinyl pyrrolidone or a composition comprising polyvinyl pyrrolidone being the most preferred super disintegrant, other useful but lesser effective disintegrants include microcrystalline cellulose and starch.

In accordance with the present invention, the type and quantity of non-active ingredients, including the binder/filler and the disintegrant or super disintegrant, are selected so as to provide a nutritional supplement tablet that dissolves in the mouth of the patient in less than about 90 seconds, preferably in less than about 80 seconds, more preferably in less than about 70 seconds, still more preferably in less than about 60 seconds, even more preferably in less than about 45 seconds, and most preferably in less than about 30 seconds after administration of the tablet. Preferably, the dissolution rate is attained without the contemporaneous administration of exogenous liquids to the patient. The tablet dissolves upon contact with saliva and agitation in the buccal cavity of the patient (e.g., by movement of the tongue) without mastication of the tablet. That is, the tablet is held in the mouth of the patient, similar to a lozenge, and not chewed.

In certain embodiments, the binder/filler and the disintegrant or super disintegrant may be provided as a single ingredient component. For example, in certain embodiments of the tablet described herein, the mannitol binder and filler is provided as a mixture that also contains a percentage of polyvinyl pyrrolidone. By way of example, the binder and filler and super disintegrant may be provided as a composition that comprises about 80% to 100%, and preferably about 90% by weight mannitol, about 0.5% to about 5%, and preferably about 5% by weight polyvinyl acetate, and about 3% to about 7%, and preferably about 5% by weight crospovidone (polyvinyl pyrrolidone). One example of such a composition is LUDIFLASH® (BASF, Switzerland), as described U.S. Patent Application Publication No. US 2008/0299194, the content of which is hereby incorporated herein by reference. Such a composition is generally in the form of a co-processed agglomerate, typically comprising d-mannitol, crospovidone, polyvinyl acetate, and povidone, and having exceptional tableting and disintegration properties that are superior to the simple mixing of a binder/filler and disintegrant or super disintegrant, and will have a controlled particle size distribution, particle structure, high bulk density, and good flowability. Generally, a combined binder/filler and disintegrant or super disintegrant composition may have a particle size distribution wherein particles having a size of greater than about 0.4 mm may constitute a maximum of 20% of the composition; particles having a size less than about 0.2 mm may constitute a maximum of about 90% to a minimum of about 45% of the composition; and particles having a size of less than about 0.063 mm may constitute a maximum of about 90% to a minimum of about 45% of the composition. In one embodiment, the combined binder/filler and disintegrant or super disintegrant component comprises between about 50% to about 80% by weight of the tablet, still more preferably from about 70% to about 80% by weight, and most preferably from about 75% to about 80% by weight of the tablet.

Moreover, although not critical, particle size of all components of the tablet (e.g., the active and non-active ingredient components) may generally be of a size that allows for direct compression of all components, and the desired hardness, friability, and dissolution or disintegration time. Typically, such components may be of a similar size, such as for example, within two orders of magnitude.

The tablet of the present invention may further comprise a lubricant. The lubricant aids in removal of the tablet from the device utilized to make the tablet, such as, for example, a tablet press. Examples of lubricants include stearic acid, magnesium stearate, and combinations of thereof. Typically, the lubricant will be present in an amount sufficient to enable removal of the tablet from the tablet press, but in an amount sufficiently minimal enough to not negatively interfere with the manufacture, disintegration characteristics, or nutritional efficacy of the tablet. Accordingly, the lubricant component of the tablet may comprise from about 0.5% to about 5% by weight of the tablet. More preferably, the lubricant comprises from about 1% to about 3% by weight of the tablet. In one embodiment, the lubricant is magnesium stearate and comprises about 1% by weight of the tablet. In another embodiment, the lubricant is magnesium stearate and comprises about 2.4% by weight of the tablet. In another embodiment, the lubricant is stearic acid and comprises about 1% by weight of the tablet. In another embodiment, the lubricant is a combination of magnesium stearate and stearic acid and comprises about 2% by weight of the tablet. In another embodiment, the lubricant is a combination of magnesium stearate and stearic acid and comprises about 3.4% by weight of the tablet.

The tablet of the present invention may further comprise a sweetener or flavoring agent. These ingredients mask the otherwise unpleasant taste of the active ingredient component, giving the composition a sweet and pleasant flavor or taste. Examples of such ingredients include, for example, mannitol, sucralose, xylitol, potassium acesulfame, fructose, ascorbic acid, and fruit punch or orange flavorings. The sweetener or flavoring agent will be present in an amount sufficient to provide a pleasing taste to the tablet, thereby improving patient compliance with the administration regimen, but not in an amount that negatively interferes with the manufacture or desired disintegration characteristics of the tablet. Typically, these ingredients will comprise about 0.5% to about 5% by weight of the tablet.

In a particular embodiment, the rapidly dissolvable tablet has the non-active ingredient component formulation disclosed in Table 3 and is suitable for preparation of a rapidly dissolving tablet for bariatric patients. In another embodiment, the rapidly dissolvable tablet has the non-active ingredient component formulation disclosed in Table 4 and is suitable preparation of a rapidly dissolving tablet for renal or dialysis patients.

TABLE 3
Tablet Excipients

| | | % wt |
|---|---|---|
| Ingredient | Function | Tablet |
| LUDIFLASH ® | Filler, binder, disintegrant | 78.31 |
| (89.5% mannitol, 3% | | |
| crospovidone, and | | |
| 1.5% PVA) | | |
| Crospovidone | Disintegrant | 1.00 |
| Stearic Acid | Lubricant for tableting process | 2.40 |
| Magnesium Stearate | Lubricant for tableting process | 1.00 |
| Sucralose | Sweetener | 0.70 |
| Flavoring agent | Flavor | 2.00 |

TABLE 4
Tablet Excipients

| | | % wt |
|---|---|---|
| Ingredient | Function | Tablet |
| LUDIFLASH ® | Filler, binder, disintegrant | 78.69 |
| (89.5% mannitol, 3% | | |
| crospovidone, and | | |
| 1.5% PVA) | | |
| Crospovidone - | Disintegrant | 1.00 |
| Kollidon CL-SF | | |
| Stearic Acid | Lubricant for tableting process | 2.40 |
| Magnesium Stearate | Lubricant for tableting process | 1.00 |
| Sucralose | Sweetener | 0.70 |
| Flavoring agent | Flavor | 0.3 |

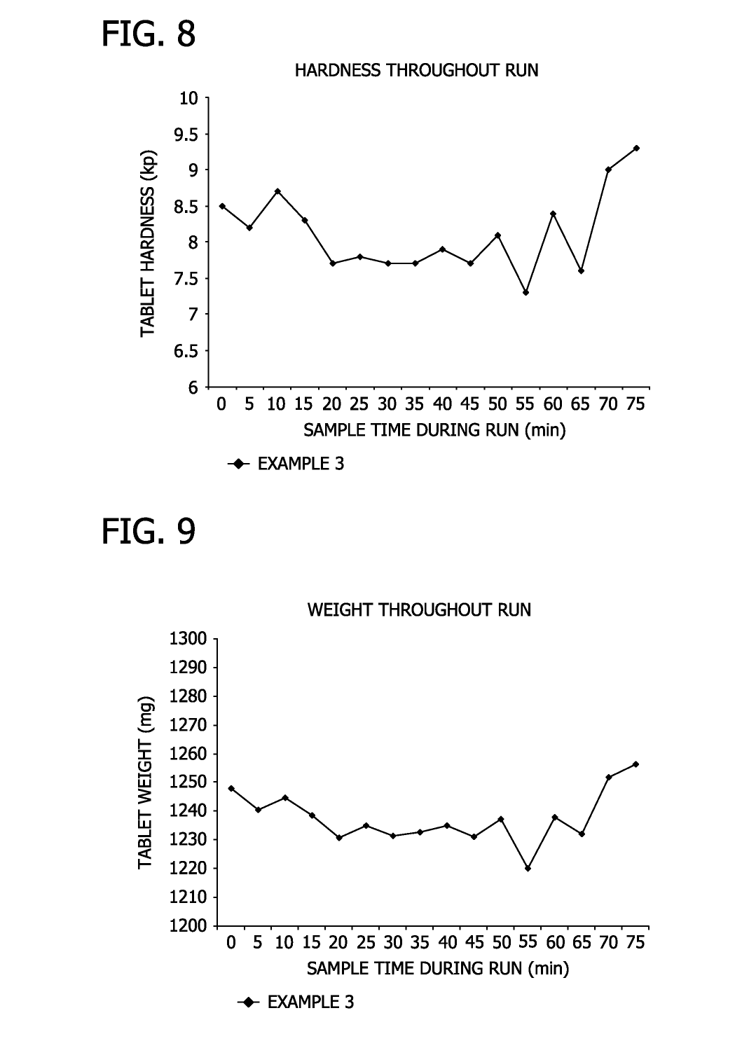

In a particular embodiment, the rapidly dissolvable nutritional composition in a two tablet format suitable for bariatric patients comprises the active and non-active ingredient component formulation disclosed in Table 4. In another particular embodiment, the rapidly dissolvable nutritional composition in a single tablet format suitable for renal or dialysis patients comprises the active and non-active ingredient component formulation disclosed in Table 5. In another particular embodiment, the rapidly dissolvable nutritional composition in a single tablet format suitable for renal or dialysis patients comprises the active and non-active ingredient component formulation disclosed in Table 6. The embodiments in Tables 6 and 7 are demonstrated as a 45 kg batch in FIG. 8.

TABLE 5
Active and non-active ingredient component formulation
of one embodiment of the rapidly dissolvable tablet

| | | | | daily dose = | | | | | |
|---|---|---|---|---|---|---|---|---|---|
| | | | | 2 tablets | | | dose + | | |
| | | Eq. of Active | | daily | | % | overage | wt % in | total weight |
| Ingredient | Purpose/Active | or Purity | units | dose | units | overage | (mg) | formulation | (mg)/tablet |
| LUDIFLASH ® | filler, binder, | 100 | % | 2132.07 | mg | | 1962.73 | 78.31 | 981.36 |
| | disintegrant | | | | | | | | |
| Mannitol premix | QS agent | 100 | % | 22.42 | mg | | 22.42 | 0.90 | 11.21 |
| | for premix | | | | | | | | |
| Ascorbic Acid | Vitamin C | 100 | % | 60 | mg | 10 | 66.00 | 2.64 | 33.00 |
| Zinc Citrate 31% | Zinc | 31 | % | 15 | mg | | 48.39 | 1.94 | 24.19 |
| Crospovidone - Kollidon CL-SF | Disintegrant | 100 | % | 25.00 | mg | | 25.00 | 1.00 | 12.50 |
| D-Biotin 1% | Biotin | 1 | % | 0.3 | mg | | 30.00 | 1.20 | 15.00 |
| Cyanocobalamin 1% | Vitamin B12 | 1 | % | 0.25 | mg | 20 | 30.00 | 1.20 | 15.00 |
| d-alpha-tocopheryl acid | Vitamin E | 1115 | IU/g | 26.9 | mg | 2 | 27.44 | 1.10 | 13.72 |
| succinate | | | | | | | | | |
| Stearic Acid | Lubricant | 100 | % | 60.00 | mg | | 60.00 | 2.40 | 30.00 |
| Iron Fumarate 31.4% | Iron | 31.41 | % | 8 | mg | | 25.47 | 1.02 | 12.73 |
| Niacinamide 99.49% | Vitamin B3/ | 99.499 | % | 20 | mg | 10 | 22.11 | 0.88 | 11.06 |
| | Niacinamide | | | | | | | | |
| Copper Citrate | Copper | 35 | % | 2 | mg | | 5.71 | 0.23 | 2.86 |
| N-C Nat & Art Fruit Punch Flav | Flavor | 100 | % | 50.00 | mg | | 50.00 | 2.00 | 25.00 |
| Calcium Pantothenate 91.74% | Vitamin B5, | 91.74 | % | 10 | mg | 15 | 12.54 | 0.50 | 6.27 |
| | Pantothenoic | | | | | | | | |
| | Acid | | | | | | | | |
| Selenium Amino Acid Chelate | Selenium | 0.2 | % | 0.055 | mg | | 27.50 | 1.10 | 13.75 |
| Mag Stearate | Lubricant | 100 | % | 25.00 | mg | | 25.00 | 1.00 | 12.50 |
| Sucralose | Sweetener | 100 | % | 12.50 | mg | | 12.50 | 0.70 | 6.25 |
| Pyridoxine HCl 82.3% | Vitamin B6 | 82.3 | % | 5 | mg | 5 | 6.38 | 0.26 | 3.19 |
| Thiamine HCl | Vitamin B1 | 100 | % | 3 | mg | 5 | 3.15 | 0.13 | 1.58 |
| Manganese Citrate 30.4% | Manganese | 30.4 | % | 2 | mg | | 6.58 | 0.26 | 3.29 |
| Riboflavin 96.5 | Vitamin B2 | 96.5 | % | 1.7 | mg | 5 | 1.85 | 0.07 | 0.92 |
| Folic Acid 90% | Folate | 90 | % | 0.6 | mg | 10 | 0.73 | 0.03 | 0.37 |
| Cholecalciferol 100,000 IU/g | Vitamin D3 | 100000 | IU/g | 8.0 | mg | 35 | 10.80 | 0.43 | 5.40 |
| Chromium Chelate 10% | Chromium | 10 | % | 0.12 | mg | | 1.20 | 0.05 | 0.60 |
| Vitamin A Acetate 500,000 | Vitamin A | 500000 | IU/g | 10.0 | mg | 35 | 13.50 | 0.54 | 6.75 |
| Molybdenum Chelate 2.5% | Molybdenum | 2.5 | % | 0.075 | mg | | 3.00 | 0.12 | 1.50 |
| | | | Total | 2500.00 | mg | | 2500.00 | 100.00 | 1250.00 |

TABLE 6
Active and non-active ingredient component formulation of one
embodiment of the rapidly dissolvable tablet

| | | | | daily dose = | | | | | |
|---|---|---|---|---|---|---|---|---|---|
| | | | | 1 tablet | | | dose + | | |
| | | Eq. of Active | | daily | | % | overage | wt % in | total weight |
| Ingredient | Purpose/Active | or Purity | units | dose | units | overage | (mg) | formulation | (mg)/tablet |
| LUDIFLASH ® | filler, binder, | 100 | % | 1070.79 | mg | | 983.64 | 78.96 | 983.64 |
| | disintegrant | | | | | | | | |
| Mannitol premix | QS agent | 100 | % | 11.2 | mg | | 11.20 | 0.90 | 11.20 |
| | for premix | | | | | | | | |
| Ascorbic Acid | Vitamin C | 100 | % | 18 | mg | 35 | 24.30 | 1.94 | 24.30 |
| Sodium Ascorbate | Vitamin C | 88.944 | % | 42 | mg | 35 | 63.75 | 5.10 | 63.75 |
| Crospovidone - Kollidon CL-SF | disintegrant | 100 | % | 12.50 | mg | | 12.50 | 1.00 | 12.50 |
| D-Biotin 1% | Biotin | 1 | % | 0.3 | mg | 35 | 40.50 | 3.24 | 40.50 |
| Cyanocobalamin 1% | Vitamin B12 | 1 | % | 0.006 | mg | 35 | 0.81 | 0.06 | 0.81 |
| Stearic Acid | lubricant | 100 | % | 30.00 | mg | | 30.00 | 2.40 | 30.00 |
| Niacinamide 99.49% | Vitamin B3/ | 99.499 | % | 20 | mg | 35 | 27.14 | 2.17 | 27.14 |
| | Niacinamide | | | | | | | | |
| Flavor | Flavor | 100 | % | 3.75 | mg | | 3.75 | 0.30 | 3.75 |
| Calcium Pantothenate 91.74% | Vitamin B5, | 91.74 | % | 5 | mg | 35 | 7.36 | 0.59 | 7.36 |
| | Pantothenoic | | | | | | | | |
| | Acid | | | | | | | | |
| Mag Stearate | lubricant | 100 | % | 12.50 | mg | | 12.50 | 1.00 | 12.50 |
| Sucralose | Sweetener | 100 | % | 8.75 | mg | | 8.75 | 0.70 | 8.75 |
| Pyridoxine HCl 82.3% | Vitamin B6 | 82.3 | % | 10 | mg | 35 | 16.40 | 1.31 | 16.40 |
| Thiamine HCl | Vitamin B1 | 100 | % | 1.5 | mg | 35 | 2.03 | 0.16 | 2.03 |
| Riboflavin 96.5 | Vitamin B2 | 96.5 | % | 1.7 | mg | 35 | 2.38 | 0.19 | 2.38 |
| Folic Acid 90% | Folate | 90 | % | 2 | mg | 35 | 3.00 | 0.24 | 3.00 |
| | | | Total | 1250.00 | mg | | 1250.00 | 100.00 | 1250.00 |

TABLE 7
Active and non-active ingredient component formulation of one
embodiment of the rapidly dissolvable tablet

| | | | | daily dose = | | | | | |
|---|---|---|---|---|---|---|---|---|---|
| | | Eq. | | 1 tablet | | | | | |
| | | of Active | | daily | | % | dose + overage | wt % in | total weight |
| Ingredient | Purpose/Active | or Purity | units | dose | units | overage | (mg) | formulation | (mg)/tablet |
| LUDIFLASH ® | filler, binder, | 100 | % | 1055.79 | mg | | 935.23 | 74.82 | 935.25 |
| | disintegrant | | | | | | | | |
| Mannitol premix | QS agent | 100 | % | 11.2 | mg | | 11.20 | 0.90 | 11.20 |
| | for premix | | | | | | | | |
| Ascorbic Acid | Vitamin C | 100 | % | 18 | mg | 35 | 24.30 | 1.94 | 24.30 |
| Sodium Ascorbate | Vitamin C | 88.944 | % | 42 | mg | 35 | 63.75 | 5.10 | 63.75 |
| Crospovidone - Kollidon CL-SF | disintegrant | 100 | % | 12.50 | mg | | 12.50 | 1.00 | 12.50 |
| D-Biotin 1% | Biotin | 1 | % | 0.3 | mg | 35 | 40.50 | 3.24 | 40.50 |
| Cyanocobalamin 1% | Vitamin B12 | 1 | % | 0.006 | mg | 35 | 0.81 | 0.06 | 0.81 |
| Stearic Acid | lubricant | 100 | % | 30.00 | mg | | 30.00 | 2.40 | 30.00 |
| Niacinamide 99.49% | Vitamin B3/ | 99.499 | % | 20 | mg | 35 | 27.14 | 2.17 | 27.14 |
| | Niacinamide | | | | | | | | |
| Flavor | Flavor | 100 | % | 3.75 | mg | | 3.75 | 0.30 | 3.75 |
| Calcium Pantothenate 91.74% | Vitamin B5, | 91.74 | % | 5 | mg | 35 | 7.36 | 0.59 | 7.36 |
| | Pantothenoic | | | | | | | | |
| | Acid | | | | | | | | |
| Mag Stearate | lubricant | 100 | % | 12.50 | mg | | 12.50 | 1.00 | 12.50 |
| Sucralose | Sweetener | 100 | % | 8.75 | mg | | 8.75 | 0.70 | 8.75 |
| Pyridoxine HCl 82.3% | Vitamin B6 | 82.3 | % | 10 | mg | 35 | 16.40 | 1.31 | 16.40 |
| Thiamine HCl | Vitamin B1 | 100 | % | 1.5 | mg | 35 | 2.03 | 0.16 | 2.03 |
| Riboflavin 96.5 | Vitamin B2 | 96.5 | % | 1.7 | mg | 35 | 2.38 | 0.19 | 2.38 |
| Zinc Citrate 31% | Zinc | 31 | % | 15 | mg | | 48.39 | 3.87 | 48.39 |
| Folic Acid 90% | Folate | 90 | % | 2 | mg | 35 | 3.00 | 0.24 | 3.00 |
| | | | Total | 1250.00 | mg | | 1250.00 | 100.00 | 1250.00 |

TABLE 8
Active and non-active ingredient component formulation for a
45 kg batch of one embodiment of the rapidly dissolvable tablet

| R# | Ingredient | % wt/wt | kg/batch | kg for 5 batches |
|---|---|---|---|---|
| 360 | Ludiflash | 74.8 | 34 | 168.35 |
| 364 | PreMix | 19.8 | 8.9 | 44.50 |
| 359 | Crospovidone | 1.0 | 0.45 | 2.25 |
| 356 | Steric Acid | 2.4 | 1.08 | 5.40 |
| 358 | Flavor | 0.3 | 0.135 | 0.68 |
| 238 | Mag Stearate | 1.0 | 0.45 | 2.25 |
| 357 | Sucralose | 0.7 | 0.315 | 1.58 |
| | Total | 100.0 | 45 | 225.00 |
| | | Batch size | 45 | |

In another particular embodiment, the rapidly dissolvable nutritional composition in a two tablet format suitable for a bariatric patient comprises the active and non-active ingredient formulation disclosed in Table 9. Each tablet of this embodiment is designed to have a smooth mouth feel and is approximately 1250 mg in weight with an off white color and fruit flavor, with a standard dosage being two tablets per day.

TABLE 9
Active and non-active ingredient formulation of one embodiment of the
rapidly dissolvable tablet

| | | Total | Total |
|---|---|---|---|
| | | Weight | Weight (g) |
| | Weight % in | (mg)/ | per 45 kg |
| Ingredient | Formulation | Tablet | Batch |
| LUDIFLASH ® | 78.31 | 978.86 | 35239.10 |
| Mannitol, USP premix | 0.90 | 11.21 | 403.56 |
| Ascorbic Acid, USP | 2.64 | 33.00 | 1188.00 |
| Zinc Citrate | 1.94 | 24.19 | 870.97 |
| Crospovidone, NF - Kollidon CL-SF | 1.00 | 12.50 | 450.00 |
| D-Biotin | 1.20 | 15.00 | 540.00 |
| Cyanocobalamin, USP | 1.20 | 15.00 | 540.00 |
| d-alpha-tocopheryl acid succinate | 1.10 | 13.72 | 493.99 |
| Stearic Acid, NF | 2.40 | 30.00 | 1080.00 |
| Iron Fumarate | 1.02 | 12.73 | 458.45 |
| Niacinamide | 0.88 | 11.06 | 397.99 |
| Copper Citrate | 0.23 | 2.86 | 102.86 |
| N-C Nat & Art Fruit Punch Flav | 2.00 | 25.00 | 900.00 |
| Calcium Pantothenate, USP | 0.50 | 6.27 | 225.64 |
| Selenium Amino Acid Chelate | 1.10 | 13.75 | 495.00 |
| Mag Stearate, NF | 1.00 | 12.50 | 450.00 |
| Sucralose, NF | 0.70 | 8.75 | 315.00 |
| Pyridoxine HCl, USP | 0.26 | 3.19 | 114.82 |
| Thiamine HCl, USP | 0.13 | 1.58 | 56.70 |
| Manganese Citrate | 0.26 | 3.29 | 118.42 |
| Riboflavin | 0.07 | 0.92 | 33.30 |
| Folic Acid, USP | 0.03 | 0.37 | 13.20 |
| Cholecalciferol, USP | 0.43 | 5.40 | 194.40 |
| Chromium Chelate | 0.05 | 0.60 | 21.60 |
| Vitamin A Acetate, USP | 0.54 | 6.75 | 243.00 |
| Molybdenum Chelate | 0.12 | 1.50 | 54.00 |

In another particular embodiment, the rapidly dissolvable nutritional composition in a two tablet format (i.e., a daily does is to be administered as two tablets) suitable for bariatric patients comprises the active and non-active ingredient formulation disclosed in Table 10.

TABLE 10
Active and non-active ingredient formulation of one embodiment of the
rapidly dissolvable tablet

| | | | Total |
|---|---|---|---|
| | | Total | Weight (g) |
| | Weight % in | Weight (mg)/ | per 6 kg |
| Ingredient | Formulation | Tablet | Batch |
| Mannitol | 73.80 | 754.10 | 4427.71 |
| Mannitol premix | 1.10 | 11.21 | 65.79 |
| Ascorbic Acid | 3.23 | 33.00 | 193.76 |
| Zinc Citrate | 2.37 | 24.19 | 142.05 |
| Crospovidone | 2.20 | 22.50 | 132.11 |
| D-Biotin | 1.47 | 15.00 | 88.07 |
| Cyanocobalamin | 1.47 | 15.00 | 88.07 |
| d-alpha-tocopheryl acid | 1.34 | 13.72 | 80.57 |
| succinate | | | |
| Steric Acid | 2.42 | 24.78 | 145.50 |
| Iron Fumarate | 1.25 | 12.73 | 74.77 |
| Niacinamide | 1.08 | 11.06 | 64.91 |
| Copper Citrate | 0.28 | 2.86 | 16.78 |
| N-C Nat & Art Fruit Punch | 1.96 | 20.00 | 117.43 |
| Flav | | | |
| Calcium Pantothenate | 0.61 | 6.27 | 36.80 |
| Selenium Amino Acid Chelate | 1.35 | 13.75 | 80.73 |
| Mag Stearate | 0.98 | 10.00 | 58.72 |
| Sucralose | 0.78 | 8.00 | 46.97 |
| Pyridoxine HCl | 0.32 | 3.31 | 19.43 |
| Thiamine HCl | 0.15 | 1.58 | 9.25 |
| Manganese Citrate | 0.32 | 3.29 | 19.31 |
| Riboflavin | 0.09 | 0.92 | 5.43 |
| Folic Acid | 0.04 | 0.37 | 2.15 |
| Cholecalciferol | 0.53 | 5.40 | 31.71 |
| Chromium Chelate | 0.06 | 0.60 | 3.52 |
| Vitamin A Acetate | 0.66 | 6.75 | 39.63 |
| Molybdenum Chelate | 0.15 | 1.50 | 8.81 |

Rapidly Dissolving Tablet—pH

The size-limited digestive anatomy of bariatric patients following surgery is susceptible to reduced production and/or availability of digestive acids. As a result, nutritional supplementation should generally account for this phenomenon. Moreover, such supplements have to account for general solubility of the ingredients of the nutritional composition as well.

Typically, therefore, although not necessarily required, the rapidly dissolving nutritional tablet will generally have a pH that is not excessively basic, and more preferably a pH that is acidic. Accordingly, in a preferred embodiment, the rapidly dissolving nutritional supplement will exhibit a pH that is from about 3 to about 10. In accordance with a more preferred embodiment, the rapidly dissolving nutritional tablet exhibits a pH of less than about 7 and more preferably, from about 3 to about 6, still more preferably from about 4 to about 5, and most preferably about 4.5. The pH of the nutritional tablet can be adjusted by proper selection of the forms of active and non-active ingredients, e.g., selection of citrate forms instead of carbonate forms of the various ingredients. Generally, pH of the rapidly dissolving nutritional tablet is determined by dissolving a tablet in approximately 100 mL of purified water and measuring the pH of the resulting solution (e.g., using a pH meter).

Rapidly Dissolving Tablet—Manufacture

The rapidly dissolvable nutritional tablet may be prepared in accordance with known manufacturing techniques. For example, a premix of the active ingredient component may be dry mixed with the non-active ingredient component in a suitable blender to form a pre-tablet mixture. This mixture is then introduced into a suitable tableting device and compressed to form the tablet.

In one embodiment, the manufacturing process utilizes the following raw materials (Table 11) and the following equipment (Table 12):

TABLE 11
Raw Materials for Tablet Manufacturing

| | Total Weight (kg) |
|---|---|
| Raw Material | per 45 kg Batch |
| LUDIFLASH ® | 35.24 |
| Crospovidone, NF - KOLLIDON ® CL-SF | 0.45 |
| Stearic Acid, NF | 1.08 |
| Fruit Punch Flavor, N-C Natural and Artificial | 0.90 |
| Magnesium Stearate, NF | 0.45 |
| Sucralose, NF | 0.32 |
| Vitamin Premix | 6.57 |

TABLE 12
Equipment for Tablet Manufacturing
Equipment

5 cubic ft V-Blender
Tablet Press
Tablet Deduster

Blending and formation of the tablet may be accomplished according to the following blending and tableting process.

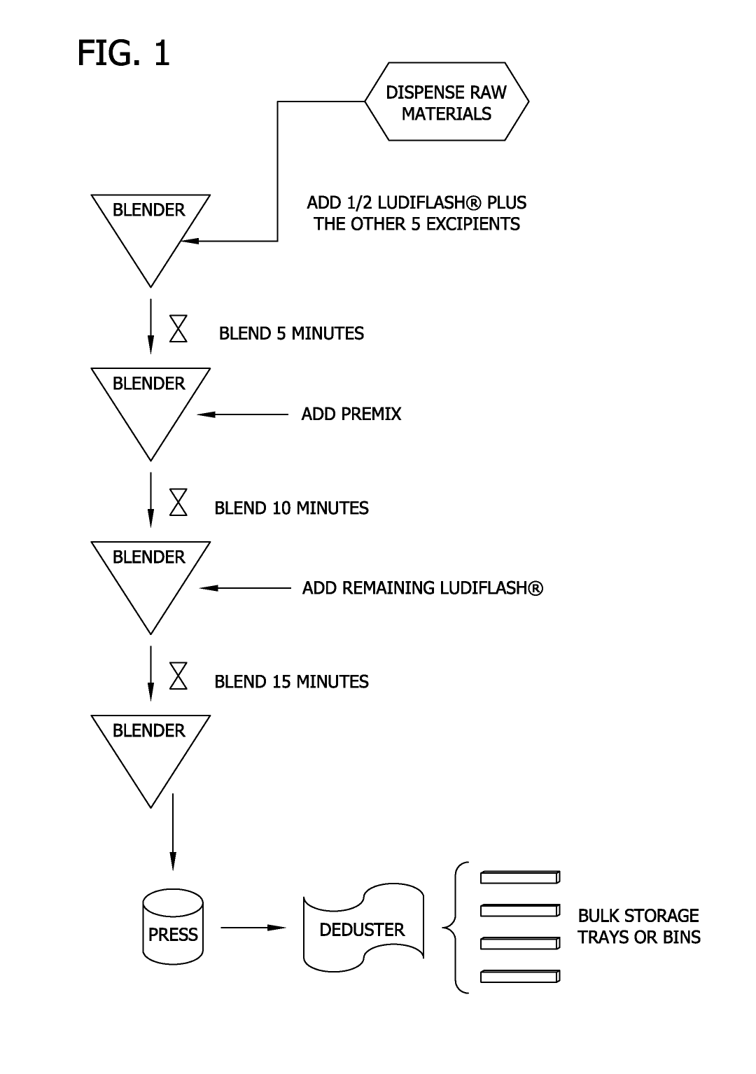

An example of a process for manufacturing a rapidly dissolvable tablet in accordance with the present invention is shown in FIG. 1. The required non-active raw materials (see Table 11) are dispensed into a blender. As shown in FIG. 1, only a portion of the binder/filler and disintegrant (e.g., LUDIFLASH®) is initially mixed with the other non-active raw materials. That is, the LUDIFLASH® is generally dispensed appropriately for two-stage addition. The order of addition may be as desired, but generally is determined based on efficiency of manufacturing. The non-active raw materials are blended for an amount of time sufficient to provide a substantially homogeneous mixture (e.g., about 5 minutes). The active ingredient component may then be added to the non-active raw material mixture as a premix and further blended for an amount of time to again provide a substantially homogeneous mixture (e.g., about 10 minutes). The remainder of the binder/filler and disintegrant (e.g., LUDIFLASH®) is added to the mixture in the blender, and this final mixture is further blended for an amount of time to again provide a substantially homogeneous mixture (e.g., about 15 minutes). The final mixture is fed to a tablet press. The target weight of each tablet is approximately 1250 mg+/−50 mg. In one embodiment, the target hardness will be about 8 kp. The tablets exiting the press are preferably dedusted before storage or packaging in bulk.

Rapidly Dissolving Tablet—Hardness and Friability

The rapidly dissolving tablet of the present invention must not only dissolve rapidly upon administration to the patient, but it must also be sufficiently attrition-resistant and capable of withstanding packaging and shipment. Accordingly, the rapidly dissolving tablet preferably exhibits a hardness of about 3 kp to about 12 kp, more preferably about 6 kp to about 10 kp, and most preferably about 8 kp.

The rapidly dissolvable tablet may exhibit a range of different friabilities depending in large part on the manner of packaging in which the tablets will be placed for storage, and in particular for shipping, and/or sale. Generally, the tablets will exhibit a friability of less than about 5%, preferably less than about 4%, more preferably less than about 3%, still more preferably less than about 2%, and most preferably less than about 1%, particularly if the tablets are manufactured for multi-dose packaging (e.g., multiple tablets packaged in a typical plastic bottle for pills or tablets). By way of example, if the tablets were to be packaged and/or sold in a typical plastic bottle used for vitamin or medicinal tablets, the friability of the tablets would tend to be lower (e.g., less than about 2% friability and more preferably less than about 1% friability).

Alternatively, the tablets may exhibit a friability from about 5% to about 75%, more preferably about 5% to about 50%, still more preferably about 5% to about 25%, and most preferably about 5% to about 10%, particularly if the tablets are manufactured for single dose packaging (e.g., tablets packaged in a typical blister pack used for pills or tablets). Similarly, tablets packaged in such a fashion may also have a lower friability, such as, for example, less than about 4% friability, preferably between about 2% and about 4% friability, and more preferably between about 3% and about 4% friability.

As tablet hardness, tablet friability, and compression force used to manufacture the tablet tend to be interrelated, the exact hardness and/or friability of the tablet may be adjusted by altering the compression force of the tableting press used in the tablet manufacturing process, as well as the tablet formulation (i.e., the type and amount of active and non-active ingredients), the tablet hardness, and the friability. Tableting pressure may be adjusted by one skilled in the art based on the particular tableting apparatus being used to achieve the desired tablet hardness and friability.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

The purpose of this example is to characterize the durability of the nutritional supplement tablet over a range of hardness levels when the non-active ingredient component utilizes a binder/filler that is separate from the disintegrant.

Protocol

Tablets utilized for this example were manufactured according to the process described above and utilizing the formulation of Table 10. Specifically, the required non-active raw materials of Table 10 were dispensed into a 5 cubic ft. V blender. Only a portion of the binder/filler (i.e., the mannitol in the form of Roquette's PEARLITOL® 100 SD) was initially mixed with the other non-active raw materials. The non-active raw materials were blended until they appeared to be well mixed (i.e., for 5 minutes) in a V blender. The active ingredient components listed in Table 9 were then added to the non-active raw material mixture as a premix and further blended until it appeared to be well mixed (i.e., for 5 minutes). The remainder of the binder/filler (i.e., the mannitol in the form of Roquette's PEARLITOL® 100 SD) was added to the mixture in the blender, and this final mixture was blended until it appeared to be well mixed (i.e., for 10 minutes). The final mixture was fed into a Manesty Express Rotary 20 station tablet press and pressed into tablets having an approximate weight of 1020 mg+/−50 mg. Tablets having a target hardness of 0.5 kp, 1.0 kp, 2.0 kp, 3.0 kp, 4.0 kp, 6.0 kp, and 8.0 kp were produced by adjusting the tableting pressure to achieve the desire tablet hardness.

Tablets of various hardnesses were tested to determine the amount of time required for the tablet to disintegrate. The tablet was held in the mouth of the test patient similar to a lozenge so that it could be dissolved by the patient's saliva and agitation in the mouth (e.g., by movement of the tongue) without mastication of the tablet.

Friability was calculated as percent loss for each level using a standard friability apparatus for 100 rotations over 4 minutes.

Tablet durability was tested using a ship test. For each target hardness level, one or two bottles of 60 tablets each were utilized. The tablets were dedusted, weighed, placed in bottle with rayon and desiccant (except for 0.5 kp and 1.0 kp, which did not have sufficient room to place rayon or desiccant in the bottle). Each bottle was packaged in a 12-unit carton for shipping, submitted for ASTM D 4169 schedule 1 testing, removed from bottle, dedusted, and reweighed to determine percent loss for each bottle of tablets.

The hardness of each level was tested with a hardness tester (e.g., Pharmatron Hardness Tester, Dr. Schleuniger Pharmatron Inc, Manchester, N.H.) at the following time points: day of manufacture, 24 hours post-manufacture, 3 days post manufacture, and 7 days post manufacture.

For each level, one bottle containing 20 tablets without rayon or desiccant were distributed for simulated use testing. Tablets were dedusted, weighed, placed in bottle, subjected to “use” conditions, removed from bottle, dedusted, and reweighed to determine % loss for each bottle of tablets.

Results and Discussion

Tablet Hardness

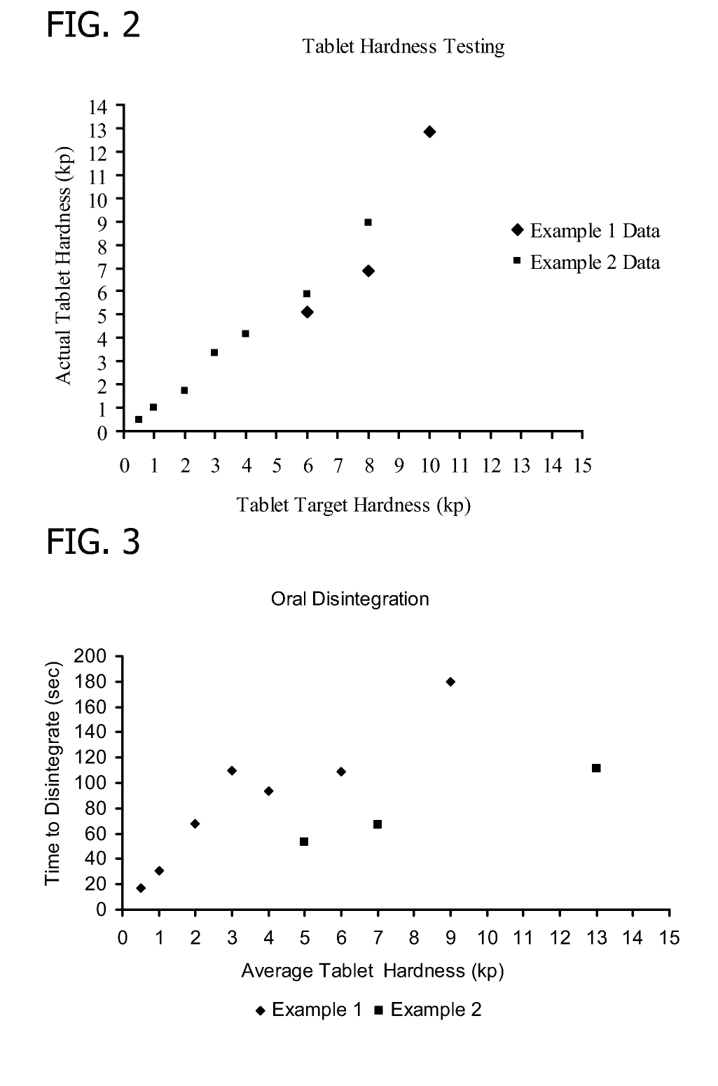

Table 13 contains tablet hardness testing information from this study. Average tablet hardness is illustrated in FIG. 2. The tablet hardness determination from this study should only be used to determine hardness levels to the nearest whole number kp value, due to the fluctuation in tablet hardness values.

TABLE 13
Results from Tablet Hardness Testing
Tablet Hardness Testing

| Tablet | | | | | | | | |
|---|---|---|---|---|---|---|---|---|
| Target | | | | | | | Average | Approximate |
| Hardness | Actual Tablet Hardness (kp) | | | | | | Tablet | # of Tablets |
| (kp) | Tablet 1 | Tablet 2 | Tablet 3 | Tablet 4 | Tablet 5 | Tablet 6 | Hardness | Manufactured |
| 0.5 | 0.5 | 0.5 | 0.4 | | | | 0.5 | 300 |
| 1.0 | 1.0 | 1.0 | | | | | 1.0 | 300 |
| 2.0 | 2.0 | 1.5 | 1.8 | 1.6 | | | 1.7 | 300 |
| 3.0 | 3.1 | 3.8 | 2.9 | 3.6 | | | 3.4 | 300 |
| 4.0 | 4.2 | 4.1 | 4.2 | | | | 4.2 | 300 |
| 6.0 | 5.6 | 5.7 | 5.1 | 6.1 | 7.0 | | 5.9 | 300 |
| 8.0 | 9.7 | 9.7 | 9.2 | 9.0 | 8.0 | 8.1 | 9.0 | 300 |

Oral Disintegration

Table 14 contains oral disintegration times for tablets from this study. Average oral disintegration times are illustrated in FIG. 3. The desired oral disintegration target of 30-90 seconds in the mouth was achieved for tablets with a hardness of 2.0 kp or less. The levels of hardness of 3.0-6.0 kp provided tablets outside this range by up to 20 seconds, while tablets with a hardness of 8.0 kp provided tablets outside this range by 90 seconds.

TABLE 14
Results from Oral Disintegration Testing
Oral Disintegration

| Tablet Target | Actual Tablet | | | |
|---|---|---|---|---|
| Hardness | Hardness (kp) | | | Average |
| (kp) | Range | Trial 1 (sec) | Trial 2 (sec) | (sec) |
| 0.5 | 0.4-0.5 | 10 | 24 | 17 |
| 1.0 | 1.0 | 33 | 28 | 30.5 |
| 2.0 | 1.5-2.0 | 86 | 49 | 67.5 |
| 3.0 | 2.9-3.8 | 159 | 61 | 110 |
| 4.0 | 4.1-4.2 | 65 | 122 | 93.5 |
| 6.0 | 5.1-7.0 | 83 | 135 | 109 |
| 8.0 | 8.0-9.7 | 100 | 260 | 180 |

Friability

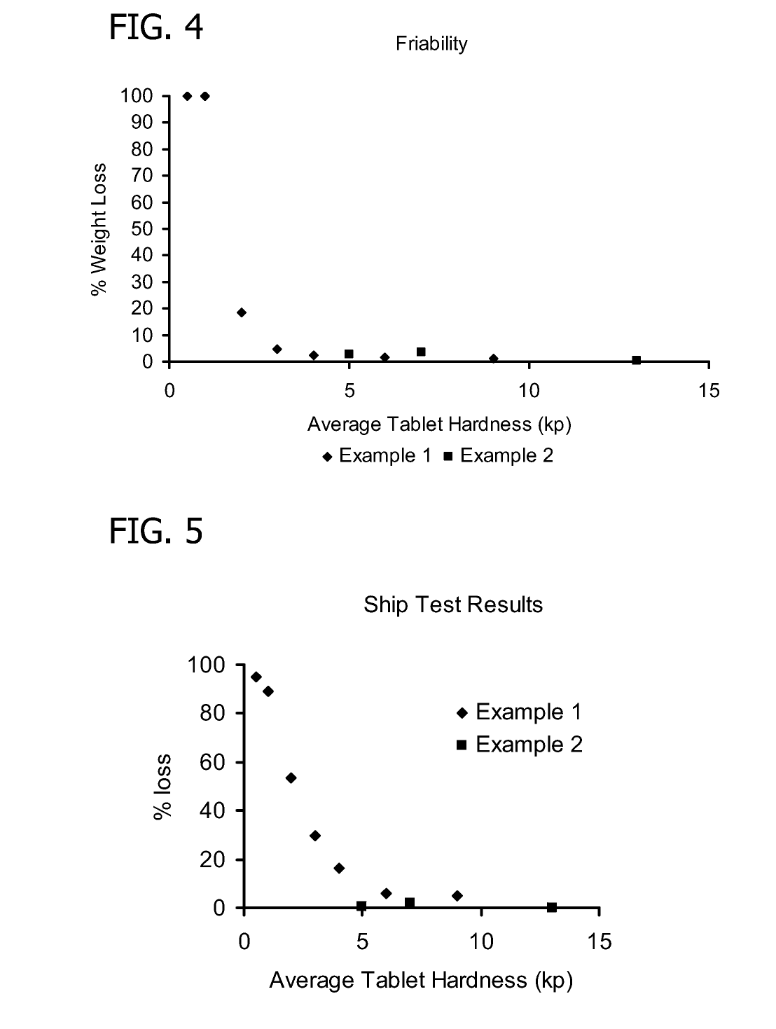

Table 15 contains results of the friability test as a percent loss. Friability as average percent loss is illustrated in FIG. 4. Hardness values of less than 3 kp yielded friability results above 5%. A hardness value of 8 kp was required to reach a friability level of 1%.

TABLE 15
Results of Friability Test
FRIABILITY

| Tablet Target | | | |
|---|---|---|---|
| Hardness | Initial Wt. of | Final Wt. of 10 | |
| (kp) | 10 Tablets (g) | Tablets (g) | % Loss |
| 0.5 | 11.3198 | 0 | 100 |
| 1.0 | 10.3466 | 0 | 100 |
| 2.0 | 10.3599 | 8.4203 | 18.7 |
| 3.0 | 10.3597 | 9.8725 | 4.7 |
| 4.0 | 10.3945 | 10.1337 | 2.4 |
| 6.0 | 10.3887 | 10.2358 | 1.5 |
| 8.0 | 10.3163 | 10.2127 | 1.0 |

Ship Test

Table 16 contains the results of the ship test from this study. Percent tablet loss as a function of tablet hardness is illustrated in FIG. 5. There was a 5% or greater loss over all tablet hardnesses during the ship test. The packaging configuration studied in this protocol (multi-dose packaging containing more than one tablet per container) is not appropriate for this formulation; however, single dose packaging (such as, for example, in a blister pack) may be a configuration that is appropriate for this formulation.

TABLE 16
Results from Ship Test
SHIP TEST

| | Initial | Final | |
|---|---|---|---|
| Tablet Target | Wt. of 60 | Wt. of 60 | |
| Hardness (kp) | Tablets (g) | Tablets (g) | % Loss |
| 0.5 | 59.9670 | 2.8976 | 95.2% |
| 1.0 Bottle-1 | 62.1360 | 3.8131 | 93.9% |
| 1.0 Bottle-2 | 62.1840 | 9.8980 | 84.1% |
| 2.0 Bottle-1 | 62.4170 | 29.0333 | 53.5% |
| 2.0 Bottle-2 | 62.4050 | 29.3415 | 53.0% |
| 3.0 Bottle-1 | 62.0630 | 41.5344 | 33.1% |
| 3.0 Bottle-2 | 62.0800 | 45.8980 | 26.1% |
| 4.0 Bottle-1 | 62.0920 | 50.6377 | 18.4% |
| 4.0 Bottle-2 | 62.3550 | 53.2810 | 14.6% |
| 6.0 | 62.0620 | 58.5070 | 5.7% |
| 8.0 Bottle-1 | 62.1310 | 58.7860 | 5.4% |
| 8.0 Bottle-2 | 61.8550 | 58.8380 | 4.9% |

Hardness Testing Over Time

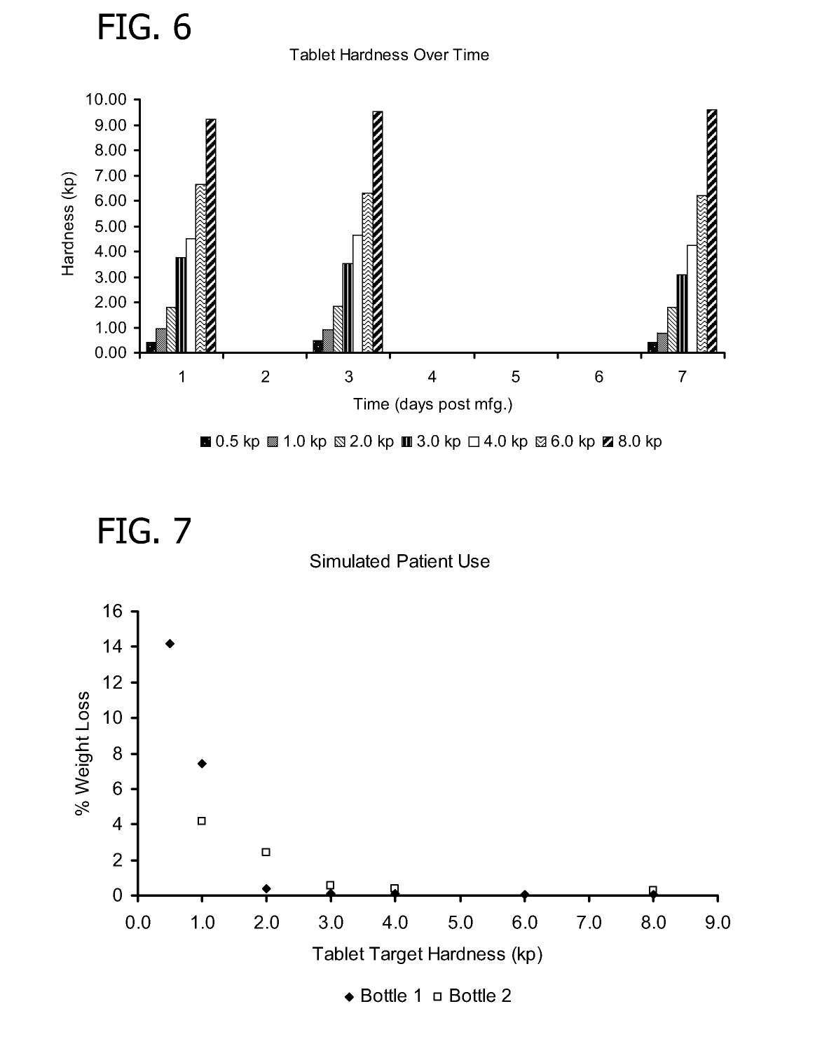

Table 17 contains tablet hardness for 24 hour, 3 day, and 7 day post-production tablets. This data is illustrated in FIG. 6. Tablet hardness remains constant at least up to one week postproduction over all tested hardness levels.

TABLE 17
Results of Tablet Hardness Over Time Testing
TABLET HARDNESS OVER TIME

| Tablet | 24 HRS POST | | | | | | | 3 DAYS POST | | | | | | | 7 DAYS POST | | | | | | |
|---|---|---|---|---|---|---|---|---|---|---|---|---|---|---|---|---|---|---|---|---|---|
| Target | MANUFACTURING | | | | | | | MANUFACTURING | | | | | | | MANUFACTURING | | | | | | |
| Hardness | | | | | | | Ave | | | | | | | Ave | | | | | | | Ave |
| (kp) | Trial 1 (kp) | | | Trial 2 (kp) | | | (kp) | Trial 1 (kp) | | | Trial 2 (kp) | | | (kp) | Trial 1 (kp) | | | Trial 2 (kp) | | | (kp) |
| 0.5 | 0.3 | 0.4 | 0.4 | 0.2 | 0.5 | 0.5 | 0.4 | 0.6 | 0.5 | 0.5 | 0.4 | 0.5 | 0.4 | 0.5 | 0.5 | 0.4 | 0.3 | 0.4 | 0.4 | 0.4 | 0.4 |
| | 0.4 | 0.4 | | 0.5 | 0.6 | | | 0.4 | 0.5 | | 0.4 | 0.4 | | | 0.3 | 0.4 | | 0.4 | 0.4 | | |
| 1.0 | 1.0 | 0.9 | 0.9 | 1.1 | 0.9 | 0.9 | 0.9 | 1.1 | 1.0 | 1.0 | 0.8 | 0.8 | 0.8 | 0.9 | 0.9 | 0.8 | 0.6 | 0.8 | 0.6 | 0.9 | 0.8 |
| | 1.0 | 1.0 | | 0.8 | 0.9 | | | 1.0 | 0.8 | | 1.0 | 0.9 | | | 0.8 | 0.8 | | 0.8 | 0.8 | | |
| 2.0 | 2.0 | 2.0 | 1.9 | 2.0 | 1.7 | 1.6 | 1.8 | 1.9 | 2.1 | 1.7 | 1.6 | 1.9 | 1.4 | 1.8 | 1.8 | 1.7 | 2.1 | 2.0 | 2.0 | 1.7 | 1.8 |
| | 1.6 | 1.7 | | 1.8 | 1.8 | | | 1.8 | 2.0 | | 1.9 | 2.0 | | | 1.6 | 1.7 | | 2.1 | 1.4 | | |
| 3.0 | 3.4 | 3.7 | 4.0 | 3.7 | 4.2 | 3.4 | 3.8 | 3.3 | 3.6 | 4.4 | 3.2 | 3.3 | 3.2 | 3.5 | 3.2 | 2.9 | 2.7 | 2.8 | 3.7 | 3.2 | 3.1 |
| | 3.5 | 3.6 | | 4.2 | 3.8 | | | 4.1 | 3.9 | | 3.2 | 3.1 | | | 2.2 | 4.0 | | 2.7 | 3.5 | | |
| 4.0 | 4.0 | 5.0 | 4.8 | 5.2 | 5.1 | 3.3 | 4.5 | 4.2 | 5.0 | 4.0 | 4.6 | 4.6 | 4.9 | 4.6 | 4.3 | 4.9 | 3.9 | 4.8 | 5.4 | 4.6 | 4.2 |
| | 4.8 | 4.3 | | 4.6 | 4.1 | | | 5.2 | 5.1 | | 4.9 | 3.8 | | | 5.0 | 0.9 | | 4.6 | 3.9 | | |
| 6.0 | 7.2 | 5.0 | 6.2 | 6.7 | 7.6 | 6.9 | 6.7 | 5.9 | 6.5 | 7.1 | 5.9 | 5.8 | 6.6 | 6.3 | 6.2 | 7.2 | 5.3 | 4.9 | 6.1 | 6.8 | 6.2 |
| | 6.7 | 7.1 | | 5.7 | 7.5 | | | 6.7 | 6.0 | | 6.2 | 6.2 | | | 7.1 | 6.6 | | 6.0 | 5.9 | | |
| 8.0 | 9.4 | 11.3 | 11.0 | 9.8 | 9.0 | 10.6 | 9.2 | 9.3 | 9.1 | 11.3 | 9.6 | 9.2 | 9.2 | 9.6 | 9.0 | 9.5 | 10.4 | 9.6 | 8.7 | 8.4 | 9.6 |
| | 5.3 | 9.8 | | 7.1 | 8.9 | | | 10.7 | 9.6 | | 7.9 | 9.2 | | | 10.2 | 10.1 | | 9.7 | 10.4 | | |

Simulated Patient Use

Table 18 contains the results of the simulated patient use test from this study. Percent tablet weight loss as a function of tablet hardness is illustrated in FIG. 7. Tablet hardness levels of 3 kp or greater experienced less than 1% loss when subjected to simulated patient use.

TABLE 18
Results for Simulated Patient Use
SIMULATED PATIENT USE

| | Initial | Final | |
|---|---|---|---|
| Tablet Target | Wt. of 20 | Wt. of 20 | |
| Hardness (kp) | Tablets (g) | Tablets (g) | % Loss |
| 0.5 | 20.6281 | 17.6897 | 14.2% |
| 1.0 Bottle-1 | 20.7754 | 19.2447 | 7.4% |
| 1.0 Bottle-2 | 20.6914 | 19.8241 | 4.2% |
| 2.0 Bottle-1 | 20.7419 | 20.6660 | 0.37% |
| 2.0 Bottle-2 | 20.8040 | 20.2960 | 2.4% |
| 3.0 Bottle-1 | 20.7319 | 20.7088 | 0.11% |
| 3.0 Bottle-2 | 20.7626 | 20.6439 | 0.57% |
| 4.0 Bottle-1 | 20.7636 | 20.7422 | 0.1% |
| 4.0 Bottle-2 | 20.7873 | 20.7117 | 0.36 |
| 6.0 | 20.6510 | 20.6456 | 0.03% |
| 8.0 Bottle-1 | 20.6660 | 20.6597 | 0.03% |
| 8.0 Bottle-2 | 20.7275 | 20.6686 | 0.28% |

Conclusions

Tablets manufactured with the formulation of Table 10 do not allow for both an average oral disintegration time of 30 to 90 seconds and a friability that allows for multi-dose packaging (although they may well allow for an average oral disintegration time of 30 to 90 seconds and a friability that allows for single dose (e.g., blister) packaging).

Tablet hardness testing has an associated percent error, which limits hardness determination to ±1 kp at best. The variability increases predictably as the hardness value increases.

### Example 2

The purpose of this example is to characterize the durability of the nutritional supplement tablet over a range of hardness levels when the non-active ingredient component utilizes a binder/filler and disintegrant that are a single component (such as, for example, LUDIFLASH® with KOLLIDON®).

Protocol

Tablets utilized for this example were manufactured according to the process described above and exemplified, for example, in FIG. 1 utilizing the formulation of Table 9. Specifically, the required non-active raw materials of Table 9 were dispensed into a 5 cubic ft. V blender. Only a portion of the binder/filler and disintegrant (i.e., the LUDIFLASH® with KOLLIDON®) was initially mixed with the other non-active raw materials. The non-active raw materials were blended until they appeared to be well mixed (i.e., for 5 minutes) in a V blender. The active ingredient components listed in Table 9 were then added to the non-active raw material mixture as a premix and further blended until it appeared to be well mixed (i.e., for 5 minutes). The remainder of the binder/filler (i.e., the LUDIFLASH® with KOLLIDON®) was added to the mixture in the blender, and this final mixture was blended until it appeared to be well mixed (i.e., for 10 minutes). The final mixture was fed into a Manesty Express Rotary 20 station tablet press and pressed into tablets having an approximate weight of 1250 mg+/−50 mg. Tablets having a target hardness of 6.0 kp, 8.0 kp, and 10 kp were produced by adjusting the tableting pressure to achieve the desire tablet hardness.

Tablets of various hardnesses were tested to determine the amount of time required for the tablet to disintegrate. The tablet was held in the mouth of the test patient similar to a lozenge so that it could be dissolved by the patient's saliva and agitation in the mouth (e.g., by movement of the tongue) without mastication of the tablet.

Friability was calculated as percent loss for each level using a standard friability apparatus for 100 rotations over 4 minutes.

Tablet durability was tested using a ship test. For each level, four bottles of 60 tablets each were dedusted, weighed, placed in a bottle with rayon, packaged in a 12 unit carton for shipping, submitted for ASTM D 4169 schedule 1 testing, removed from the bottle, dedusted, and reweighed to determine percent loss for each bottle of tablets.

Results and Discussion

Table 19 contains tablet hardness testing information from this study. Average tablet hardness is illustrated in FIG. 2. The tablet hardness determination from this study should only be used to determine hardness levels to the nearest whole number kp value, due to the fluctuation in tablet hardness values.

TABLE 19
Results from Tablet Hardness Testing
TABLET HARDNESS TESTING

| Tablet | | | | | | | | | | | | |
|---|---|---|---|---|---|---|---|---|---|---|---|---|
| Target | Actual Tablet Hardness (kp) | | | | | | | | | | Average | Approx. # of |
| Hardness | | | | | | | | | | Tab | Tablet | Tablets |
| (kp) | Tab 1 | Tab 2 | Tab 3 | Tab 4 | Tab 5 | Tab 6 | Tab 7 | Tab 8 | Tab 9 | 10 | Hardness | Manufactured |
| 6.0 | 4.9 | 5.1 | 5.1 | 5.0 | 5.3 | 5.4 | 5.8 | 4.8 | 5.0 | 4.9 | 5.1 | 400 |
| 8.0 | 8.3 | 7.7 | 7.2 | 7.4 | 4.9 | 6.3 | 4.8 | 8.9 | 5.4 | 8.1 | 6.9 | 400 |
| 10.0 | 15.4 | 11.7 | 12.0 | 13.7 | 12.9 | 11.8 | 12.1 | 12.5 | 12.9 | 13.3 | 12.8 | 400 |

Oral Disintegration

Table 20 contains oral disintegration times for tablets from this study. Average oral disintegration times are illustrated in FIG. 3. The desired oral disintegration target of 30-90 seconds in the mouth was achieved for tablets with a hardness of 8.0 kp or less. The level of hardness of 10.0 kp provided tablets outside this range by 21 seconds.

TABLE 20
Results from Oral Disintegration Testing
ORAL DISINTEGRATION

| Tablet | Actual | | | | |
|---|---|---|---|---|---|
| Target | Tablet | | | | |
| Hardness | Hardness | Trial 1 | | | Average |
| (kp) | (kp) Range | (sec) | Trial 2 (sec) | Trial 3 (sec) | (sec) |
| 6.0 | 4.8-5.8 | 54 | 45 | 60 | 53 |
| 8.0 | 4.8-8.9 | 50 | 87 | 65 | 67 |
| 10.0 | 11.7-15.4 | 121 | 100 | 113 | 111 |

Friability

Table 21 contains results of the friability test as a percent loss. Friability as average percent loss is illustrated in FIG. 4. Hardness values of 6 kp and 8 kp yielded friability results above 1%. A hardness value of 10 kp was required to reach a friability level of 1%.

TABLE 21
Results of Friability Test
FRIABILITY

| Tablet | Initial Wt. | | | |
|---|---|---|---|---|
| Hardness | of 10 | Final Wt. of | | |
| (kp) | Tablets | 10 Tablets | % Loss | Comments |
| 6.0 | 12.7028 | 12.3290 | 2.94% | Chipped and cleaved. |
| 8.0 | 12.3989 | 11.9834 | 3.35% | Broken (1), the rest had |
| | | | | chips and cleaved. |
| 10.0 | 12.7854 | 12.7206 | 0.51% | Chips |

Ship Test

Table 22 contains the results of the ship test from this study. Percent tablet loss as a function of tablet hardness is illustrated in FIG. 5. There was a less than 1% loss over the 6.0 kp and 10.0 kp tablet hardness during the ship test. The 8.0 kp tablet hardness resulted in an average 2.1% loss.

TABLE 22
Results from Ship Test
SHIP TEST

| Tablet Target | Initial Wt. of 60 | Final Wt. of 60 | |
|---|---|---|---|
| Hardness (kp) | Tablets (g) | Tablets (g) | % Loss |
| 6a | 75.9487 | 75.0245 | 1.22 |
| 6b | 76.0080 | 75.6623 | 0.45 |
| 6c | 76.1649 | 75.7503 | 0.54 |
| 6d | 76.1255 | 75.6372 | 0.64 |
| Average of 4 units | | | 0.71 |
| 8a | 72.4224 | 70.5199 | 2.63 |
| 8b | 72.8916 | 71.5377 | 1.86 |
| 8c | 72.9436 | 71.5202 | 1.95 |
| 8d | 73.2324 | 71.7921 | 1.97 |
| Average of 4 units | | | 2.10 |
| 10a | 76.6954 | 76.5804 | 0.15 |
| 10b | 76.7332 | 76.6381 | 0.12 |
| 10c | 76.8666 | 76.7841 | 0.11 |
| 10d | 76.9389 | 76.8781 | 0.08 |

| | |
|---|---|
| Average of 4 units | 0.12 |

Conclusions

Tablets manufactured with a hardness value of 6 and 8 kp with the formulation of Table 9 allows for both an average oral disintegration time of approximately 30 to 90 seconds and a friability that allows for multi-dose packaging (as well as single-dose packaging).

Tablet hardness testing has an associated percent error, which limits hardness determination to ±1 kp at best. The variability increases predictably as the hardness value increases.

### Example 3

The purpose of this example is to characterize process parameters and determine in-process tablet specification ranges for the nutritional supplement tablet.

Protocol

Tablets utilized for this example were manufactured according to the process described above and exemplified in FIG. 1 utilizing the raw materials listed in Table 23 to form the tablet detailed in Table 9. Specifically, the required non-active raw materials of Table 23 were dispensed into a 5 cubic ft. V blender. Only half of the LUDIFLASH® was initially mixed with the other five non-active raw materials (i.e., the KOLLIDON® CL-SF, stearic acid, fruit punch flavor, magnesium stearate, and sucralose). The non-active raw materials were blended for 5 minutes in a 5 ft³ V blender using the intensifier bar. The active ingredient components listed in Table 9 were then added to the non-active raw material mixture as a premix (Table 25) and further blended for 10 minutes utilizing the intensifier bar. The remainder of the LUDIFLASH® was added to the mixture in the blender, and this final mixture was blended for 15 minutes utilizing the intensifier bar. The final mixture was fed into a Manesty Express D3B rotary 16 station, single action tablet press and pressed into tablets having a weight of 1250 mg+/−50 mg (equivalent to +/−4%) and a hardness of 8 kp+/−2 kp. The tablets were approximately 0.63 inch (1.6 mm) round, off-white, uncoated tablets, with both upper and lower surfaces being plain. The tablets were dedusted.

TABLE 23
Materials for Tablet Produced According to Example 3

| | | | Quantity Required | |
|---|---|---|---|---|
| Description | mg/tablet | | per 45 kg Batch (kg) | |
| Ludiflash | 978.86 | | 35.24 | |
| Kollidon CL-SF | 12.50 | | 0.45 | |
| Stearic Acid | 30.00 | | 1.08 | |
| Fruit Punch Flavor | 25.00 | | 0.90 | |
| Magnesium Stearate | 12.50 | | 0.45 | |
| Sucralose | 8.75 | | 0.32 | |
| Premix | 182.5 | | 6.57 | |
| Total | 1250 | mg | 45.00 | kg |

The tablets were tested to determine the amount of time required for the tablet to disintegrate. The tablet was held in the mouth of the test patient similar to a lozenge so that it could be dissolved by the patient's saliva and agitation in the mouth (e.g., by movement of the tongue) without mastication of the tablet.

Friability was calculated as percent loss for each level using a standard friability apparatus for 100 rotations over 4 minutes.

Results and Discussion

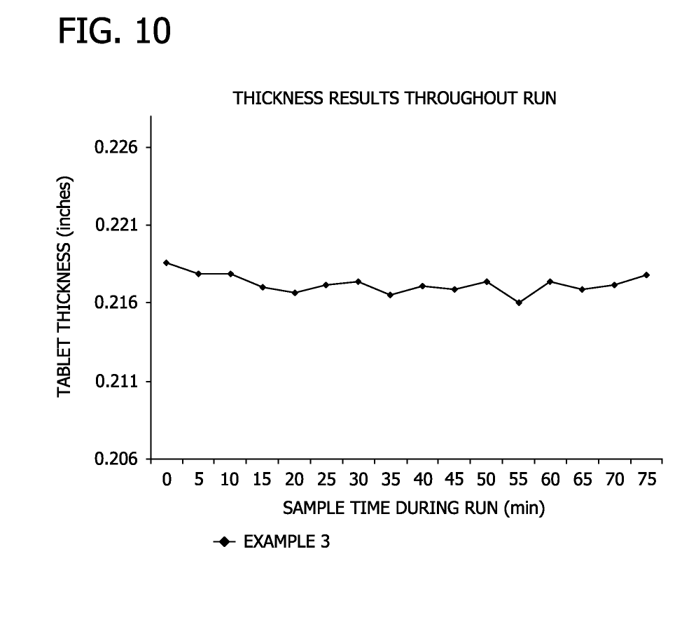

The average hardness, weight, thickness, and of the tablets produced throughout this production are illustrated in FIGS. 8, 9, and 10, respectively.

Oral Disintegration

Table 24 contains oral disintegration times for tablets from this study. The desired oral disintegration target of 30-90 seconds in the mouth was achieved for tablets, with an average time to dissolve being 65 seconds.

TABLE 24
Results from Oral Disintegration Test

| Analyst | Time to dissolve (sec) |
|---|---|
| 1 | 55 |
| 2 | 62 |
| 3 | 78 |
| Average | 65 |

Friability

Table 25 contains the results of the friability test as a percent loss. Tablets produced according to this procedure had a percent loss of 1.35%.

TABLE 25
Results from Friability Test

| | |
|---|---|
| Initial Wt of 10 Tablets | 12.4233 |
| Final Wt of 10 Tablets | 12.2560 |
| Percent Loss | 1.35% |

### Example 4

The purpose of this example is to illustrate a procedure for the preparation of a large batch of the nutritional supplement tablets for sale and patient consumption.

Protocol

Tablets utilized for this example were manufactured according to the process described above and exemplified in FIG. 1 utilizing the raw materials listed in Table 26 to form the tablet detailed in Table 9. The required non-active raw materials of Table 26 were dispensed into a 5 cubic ft. PK V blender (Patterson-Kelley, East Stroudsburg, Pa.). Specifically, the following raw materials were added to the 5 cubic ft. V blender in the following order: 18.00 kg of LUDIFLASH®, 450 g of Crospovidone, NF-KOLLIDON® CL-SF, 1080 g of stearic acid NF, 900 g of fruit punch flavor, N-C natural and artificial, 450 g of magnesium stearate NF, and 320 g of sucralose NF. The blender was activated, as was the intensifier bar, and the materials were blended for 5 minutes. 7.60 kg of the vitamin premix was added to the blender. The blender was again activated, as was the intensifier bar, and the materials were blended for 10 minutes. 16.25 kg of LUDIFLASH® was added to the blender. The blender was again activated, as was the intensifier bar, and the materials were blended for 30 minutes. The final mixed blend was discharged from the blender and collected for compression. The final product was loaded in the product hopper on a Manesty D3B Rotary Tablet Press (Manesty, Knowsley, Merseyside, UK) and pressed into tablets having a weight of 1250 mg+/−38 mg (equivalent to +/−3.04%) and a hardness of 8 kp+/−2 kp. The tablets were approximately 0.63 inch (1.6 mm) round, off-white, uncoated tablets, with both upper and lower surfaces debossed with “PBT.” The tablets were dedusted.

TABLE 26
Materials for Tablet Produced According to Example 4

| | | | Quantity | |
|---|---|---|---|---|
| | | | Required | |
| Description | mg/tablet | | per Batch | |
| LUDIFLASH ® | 951.03 | | 34.25 | kg |
| Crospovidone, NF - KOLLIDON ® CL-SF | 12.50 | | 450 | g |
| Stearic Acid, NF | 30.00 | | 1080 | g |
| Fruit Punch Flavor, N-C Natural and | 25.00 | | 900 | g |
| Artificial | | | | |
| Magnesium Stearate, NF | 12.50 | | 450 | g |
| Sucralose, NF | 8.75 | | 320 | g |
| Vitamin Premix | 210.13 | | 7.60 | kg |
| Total | 1250 | mg | 45.05 | kg |

The above description of the preferred embodiments is intended only to acquaint others skilled in the art with the invention, its principles, and its practical application, so that others skilled in the art may adapt and apply the invention in its numerous forms, as may be best suited to the requirements of a particular use. The present invention, therefore, is not limited to the above embodiments, and may be variously modified.

With reference to the use of the word(s) “comprise” or “comprises” or “comprising” in this specification, including the appended claims, unless the context requires otherwise, those words are used on the basis and clear understanding that they are to be interpreted inclusively, rather than exclusively, and that it is intended each of those words to be so interpreted in construing this specification.

### REFERENCES

USP 32-NF 27 monograph for Oil & Water Soluble Vitamins with Minerals Tablets
Jacques, Jacqueline, N D. Micronutrition for the Weight Loss Surgery Patient, Matrix Medical Communications: Edgemont, Pa., 2006.
Reavley, Nicola. The New Encyclopedia of Vitamins, Minerals, Supplements, & Herbs, Bookman Press: Austrailia, 1998.
Kurian, M. S.; Thompson, B.; Davidson, B. K. Weight Loss Surgery for Dummies, Wiley Publishing, Inc.: Indianapolis, Ind., 2005.
Griffith, H. W., MD. Vitamins, Herbs, Minerals, & Supplements the Complete Guide, Revised Edition; Da Capo Press: Cambridge, Mass., 1998.
Sutterer, A. Fleming Pharmaceuticals, Fenton, Mo. Laboratory Notebook #564 pages 1-12, 2008.
Wakefield, L. R&D Study Protocol ProBarimin QT Characterization of Tablet Durability; SN08-003; Fleming Pharmaceuticals: Fenton, Mo., 2008.
Wakefield, L. R&D Study Protocol ProBarimin QT Characterization of Tablet Durability Study 2; SN08-004; Fleming Pharmaceuticals: Fenton, Mo., 2008.
Shulman, Vic. Practical Pharmaceutical Manufacturing. AAPS Training, Toronto, Ontario Canada, September 25^{th} & 26^{th}, 2007: Academy of Applied Pharmaceutical Sciences.
LUDIFLASH® Technical Information; EMP 070805-01; BASF: www.basf.com, April 2008.
Insoluble KOLLIDON® grades Technical Information, EMP 030753e-05, BASF: www.basf.com, June 2008.
Sutterer, A. Technical Package Fleming Pharmaceuticals Product 0218, revision 4, Fleming Pharmaceuticals: Fenton, Mo., November 2008.
Rowe, Sheskey, & Weller. Handbook of Pharmaceutical Excipients, 4^{th} edition, Pharmaceutical Press: Grayslake, Ill., 2003.
Dietary Reference Intakes (DRIB): Recommended Intakes for Individuals, Elements. Food and Nutrition Board, Institute of Medicine, National Academies, 2004.
Dietary Reference Intakes (DRIB): Tolerable Upper Intake Levels (UL), Elements. Food and Nutrition Board, Institute of Medicine, National Academies, 2004.
Dietary Reference Intakes (DRIs): Recommended Intakes for Individuals, Vitamins. Food and Nutrition Board, Institute of Medicine, National Academies, 2004.
Dietary Reference Intakes (DRIs): Tolerable Upper Intake Levels (UL), Vitamins. Food and Nutrition Board, Institute of Medicine, National Academies, 2004.
Dietary Reference Intakes (DRIs): Estimated Average Requirements for Groups. Food and Nutrition Board, Institute of Medicine, National Academies, 2004.
Dietary Reference Intakes DRI (US Food and Nutrition Board) Roche Vitamins Europe Ltd, www.roche.com/vitamins/rve
Swilley, D. RD. Micronutrient and Macronuitrient Needs in Roux-en-Y Gastric Bypass Patients. Bariatric Times, March 2008, P. 1 & PP. 10-14.
Decker, G. A.; Swain, J. M; Crowell, M. C.; Scolapio, J. S. Gastrointestinal and Nutritional Complications After Bariatric Surgery. Am J. Gastroenterol. 2007; 102 (11): 2571-2580. 2007 Blackwell Publishing
Gohel, M. C.; Parikh, R. K.; Brahmbhatt, B. K.; Shah, Aarohi. Preparation and Assessment of Novel Coprocessed Superdisintegrant Consisting of Crospovidone and Sodium Starch Glycolate: A Technical Note. AAPS PharmSciTech 2007; 8 (1) Article 9 (www.aapspharmscitech.org)

1-78. (canceled)

79. A method for providing nutritional supplementation to a patient in need of the same, said method comprising orally administering to the patient a rapidly dissolving tablet, the tablet comprising:
an active ingredient component comprising one or more vitamins;
a super disintegrant; and
a directly-compressible, water-soluble mannitol;
wherein the super disintegrant and the directly compressible, water-soluble mannitol are provided as a co-processed agglomerate.

80. The method of claim 79, wherein the active ingredient component also comprises one or more minerals.

81. The method of claim 79, wherein the active ingredient comprises a mixture of two or more B vitamins selected from the group consisting of B₁, B₂, B₃, B₅, B₆, B₇, B₉, and B₁₂; and one or more non-B vitamins.

82. The method of claim of 81, wherein the active ingredient component comprises one or more non-B vitamins selected from the group consisting of vitamin A, vitamin C, vitamin D, vitamin E, and vitamin K.

83. The method of claim 82, wherein the active ingredient component comprises one or more minerals selected from the group consisting of boron, bromine, cadmium, calcium, chloride, chromium, cobalt, copper, fluoride, iodine, iron, magnesium, manganese, molybdenum, nickel, phosphorous, potassium, selenium, silicon, sodium, tin, tungsten, vanadium, and zinc.

84. The method of claim 81, wherein the rapidly dissolvable tablet is sugar free.

85. The method of claim 79, wherein the patient in need of nutritional supplementation is a patient having undergone bariatric surgery, a renal or dialysis patient, a candidate for bariatric surgery, or a candidate for dialysis.

86. The method of claim 80, wherein the patient is a candidate for bariatric surgery or a patient having undergone bariatric surgery.

87. The method of claim 86, wherein the active ingredient component of the rapidly dissolving tablet is free of therapeutic quantities of calcium and/or magnesium.

88. The method of claim 87, wherein the method further comprises the step of orally administering to the patient a second tablet, the tablet comprising a therapeutic quantity of calcium or magnesium.

89. The method of claim 86, wherein the active ingredient component comprises 1.5 mg thiamine HCl per tablet, 0.85 mg riboflavin per tablet, 10 mg niacinamide per tablet, 5 mg calcium pantothenate per tablet, 2.5 mg pyridoxine HCl per tablet, 150 μg d-biotin per tablet, 300 μg folic acid per tablet, 125 μg cyanocobalamin per tablet, 2500 IU vitamin A acetate per tablet, 30 μg ascorbic acid per tablet, 400 IU cholecalciferol per tablet, 15 IU d-alpha-tocopheryl acid succinate per tablet, 60 μg chromium chelate per tablet, 1 mg copper citrate per tablet, 4 mg iron fumarate per tablet, 1 mg manganese citrate per tablet, 37.5 μg molybdenum chelate per tablet, 27.5 μg selenium amino acid chelate per tablet, and 7.5 mg zinc citrate.

90. The method of claim 85, wherein the patient is a candidate for dialysis or a renal or dialysis patient.

91. The method of claim 90, wherein the active ingredient component of the rapidly dissolving tablet is free of phosphorous, potassium, and/or sodium.

92. The method of claim 90, wherein the active ingredient component comprises 1.5 mg thiamine HCl per tablet, 1.7 mg riboflavin per tablet, 20 mg niacinamide per tablet, 5 mg calcium pantothenate per tablet, 10 mg pyridoxine HCl per tablet, 150 μg d-biotin per tablet, 1 mg folic acid per tablet, 6 μg cyanocobalamin per tablet, and 100 mg ascorbic acid per tablet.

93. The method of claim 79, wherein the rapidly dissolving tablet dissolves in the mouth of the patient in a period of time selected from the group consisting of less than about 90 seconds, less than about 80 seconds, less than about 70 seconds, less than about 60 seconds, less than about 45 seconds, and less than about 30 seconds.

94. A rapidly dissolving tablet for oral administration for use in providing nutritional supplementation to a patient in need thereof, said tablet comprising:
an active ingredient component comprising a mixture of two or more B vitamins selected from the group consisting of B₁, B₂, B₃, B₅, B₆, B₇, B₉, and B₁₂, and one or more non-B vitamins;
a super disintegrant; and
a directly-compressible, water-soluble carbohydrate;
wherein the super disintegrant and the directly compressible, water-soluble mannitol are provided as a co-processed agglomerate.

95. The rapidly dissolving tablet of claim 94, wherein the active ingredient component is free of therapeutic quantities of calcium and/or magnesium.

96. The rapidly dissolving tablet of claim 95, wherein the active ingredient component comprises 1.5 mg thiamine HCl per tablet, 0.85 mg riboflavin per tablet, 10 mg niacinamide per tablet, 5 mg calcium pantothenate per tablet, 2.5 mg pyridoxine HCI per tablet, 150 μg d-biotin per tablet, 300 μg folic acid per tablet, 125 μg cyanocobalamin per tablet, 2500 IU vitamin A acetate per tablet, 30 μg ascorbic acid per tablet, 400 IU cholecalciferol per tablet, 15 IU d-alpha-tocopheryl acid succinate per tablet, 60 μg chromium chelate per tablet, 1 mg copper citrate per tablet, 4 mg iron fumarate per tablet, 1 mg manganese citrate per tablet, 37.5 μg molybdenum chelate per tablet, 27.5 μg selenium amino acid chelate per tablet, and 7.5 mg zinc citrate.

97. The rapidly dissolving tablet of claim 94, wherein the active ingredient component is free of phosphorous, potassium, and/or sodium.

98. The rapidly dissolving tablet of claim 97, wherein the active ingredient component comprises 1.5 mg thiamine HCl per tablet, 1.7 mg riboflavin per tablet, 20 mg niacinamide per tablet, 5 mg calcium pantothenate per tablet, 10 mg pyridoxine HCI per tablet, 150 μg d-biotin per tablet, 1 mg folic acid per tablet, 6 μg cyanocobalamin per tablet, and 100 mg ascorbic acid per tablet.
